(19) United States
(12) Patent Application Publication
(10) Pub. No.: US 2025/0261977 A1
(43) Pub. Date: Aug. 21, 2025

(54) SYSTEMS, METHODS, AND DEVICES FOR DISTRACTION HISTOGENESIS

(71) Applicant: BioDynamik, Inc., Lake Forest, CA (US)

(72) Inventors: Shanbao Cheng, Lake Forest, CA (US); Emmon Johnny Chen, Lake Forest, CA (US)

(73) Assignee: BioDynamik, Inc., Lake Forest, CA (US)

(21) Appl. No.: 19/054,197

(22) Filed: Feb. 14, 2025

Related U.S. Application Data
(60) Provisional application No. 63/554,970, filed on Feb. 17, 2024.

(51) Int. Cl. A61B 17/80; A61B 17/00

(52) U.S. Cl. CPC A61B 17/8019; A61B 2017/00221; A61B 2017/00398

(57) ABSTRACT

Systems, methods, and devices for distraction histogenesis and angiogenesis. A system includes an anchor screw comprising a hollow interior defined by a sidewall, wherein the anchor screw comprises internal actuation threading attached to an interior surface of the sidewall and further comprises external anchor threading attached to an exterior surface of the sidewall. The system includes an actuation screw comprising external actuation threading attached to an exterior surface of the actuation screw. The system includes a distraction plate coupled to the actuation screw. The system is such that the external actuation threading corresponds with the internal actuation threading such that the actuation screw is configured to be screwed into the hollow interior of the anchor screw. The system is such that the distraction plate is rotationally independent of the actuation screw. The system includes an actuation screw adjustor which can be either manual or automatic.

### TECHNICAL FIELD

The disclosure relates generally to systems, methods, and devices for surgical procedures, and relates specifically to systems, methods, and devices for distraction histogenesis procedure and angiogenesis procedures.

### BACKGROUND

In some cases, a patient benefits from improved blood flow and tissue regeneration to aid in recovery from vascular occlusion or damage, tissue trauma, bone defects, nerve damage, and other wounds. Tissue regeneration can be a major challenge for patient care and can be particularly difficult for patients experiencing ischemic diseases, chronic wounds, diabetic foot ulcers, bone defects, and other health challenges.

Tissue regeneration is particularly difficult to trigger when treating diabetic patients, and especially when treating diabetic foot ulcers. Currently, there are few known successful treatments for recalcitrant diabetic foot ulcers. Ongoing research suggests there may be some success in topical therapies and oxygen treatments, but these treatments show limited success.

Additionally, it can be difficult to successfully trigger bone regeneration in response to a bone defect, fracture, or other trauma. Commonly used therapies for bone regeneration include bone graft substitutes, guided bone regeneration (GBR), distraction osteogenesis (DO), and periosteal distraction osteogenesis (PDO). PDO is a combination of tissue expansion and GBR and is implemented to create an artificial space between the bone surface and periosteum layer by expanding the periosteum, muscle, and skin at the same time.

Traditional devices for performing distraction histogenesis and angiogenesis, and specifically for performing periosteal distraction, include numerous external components that may be bothersome and painful for the patient, can be difficult and time consuming to install, require numerous component implanted into patient bone tissue, and can be difficult to adjust as time goes on. What is needed are systems, methods, and devices for performing distraction histogenesis that improve patient outcomes, reduce surgery complexity, reduce the number of percutaneous sites, and reduce the quantity of cumbersome and painful external components, and reduce the complexity of tasks that need to be performed after surgery.

In view of the foregoing, disclosed herein are systems, methods, compositions, and devices for improved surgical procedures, and specifically for improved distraction histogenesis procedures such as periosteal distraction.

### BRIEF DESCRIPTION OF THE DRAWINGS

Non-limiting and non-exhaustive implementations of the present disclosure are described with reference to the following figures, wherein like reference numerals refer to like parts throughout the various views unless otherwise specified. Advantages of the present disclosure will become better understood with regard to the following description and accompanying drawings where:

FIG. 1A is an overhead perspective view of a system for performing a distraction histogenesis surgical procedure, wherein an actuation screw is at least partially distracted from an anchor screw;

FIG. 1B is an overhead perspective view of a system for performing a distraction histogenesis surgical procedure, wherein an actuation screw is at least partially distracted from an anchor screw;

FIG. 2 is an underside perspective view of a system for performing a distraction histogenesis surgical procedure, wherein an actuation screw is at least partially distracted from an anchor screw;

FIG. 3 is a straight-on wide view of a system for performing a distraction histogenesis surgical procedure, wherein an actuation screw is at least partially distracted from an anchor screw;

FIG. 4 is an overhead perspective view of a system for performing a distraction histogenesis surgical procedure, wherein an actuation screw is at least partially distracted from an anchor screw;

FIG. 5 is an underside perspective view of a system for performing a distraction histogenesis surgical procedure, wherein an actuation screw is at least partially distracted from an anchor screw;

FIG. 6 is an overhead perspective view of a system for performing a distraction histogenesis surgical procedure, wherein an actuation screw is fully retracted into an anchor screw;

FIG. 7 is an underside perspective view of a system for performing a distraction histogenesis surgical procedure, wherein an actuation screw is fully retracted into an anchor screw;

FIG. 8 is an aerial top-down view of a system for performing a distraction histogenesis surgical procedure, wherein an actuation screw is fully retracted into an anchor screw;

FIG. 9 is a perspective cross-sectional view of a system for performing a distraction histogenesis surgical procedure, wherein an actuation screw is fully retracted into an anchor screw;

FIG. 10 is a perspective cross-sectional view of a system for performing a distraction histogenesis surgical procedure, wherein an actuation screw is fully retracted into an anchor screw;

FIG. 11A is a schematic illustration of an exploded perspective view of an anti-rotational assembly, which is a component of a system for performing a distraction histogenesis surgical procedure;

FIG. 11B is a schematic illustration of an exploded perspective view of a system for performing a distraction histogenesis surgical procedure;

FIG. 12 is a schematic illustration of a side view of an actuator linkage;

FIG. 13 is a schematic illustration of a straight-on side view of an anchor screw;

FIG. 14 is a schematic illustration of a system for performing a distraction histogenesis surgical procedure with manual manipulation of an actuation screw;

FIG. 15 is a schematic illustration of a system for performing a distraction histogenesis surgical procedure with automated manipulation of an actuation screw;

FIG. 16 is a schematic illustration of a side view of a cannulated driver;

FIG. 17 is a schematic illustration of a perspective view of a drill sleeve;

FIGS. 18A-18E are schematic block diagrams of a method for performing a distraction histogenesis surgical procedure;

FIGS. 19A-19B are schematic block diagrams of a method for performing a distraction histogenesis surgical procedure;

FIG. 20 is a schematic block diagram of a system for monitoring health data and remotely actuating adjustment of a distraction device; and

FIG. 21 is a schematic block diagram of an example computing device.

### DETAILED DESCRIPTION

Described herein are systems, methods, and devices for triggering tissue regeneration and improving perfusion of patient fluids and releasing growth factors. The disclosures herein may be implemented to trigger regeneration of bone tissue, vascular tissue, microvascular tissue, skin tissue, nerve tissue, and other tissue types. The systems, methods, and devices described herein may be utilized to perform distraction histogenesis and angiogenesis surgical procedures on various tissue types. The systems, methods, and devices described herein may specifically be utilized to perform periosteal distraction and osteotomy procedures to trigger regeneration of soft tissue.

A system described herein is designed for performing a distraction histogenesis procedure on a patient. The system includes a distraction plate configured to be disposed in between a periosteum and an outer surface of a bone. The system includes an anchor screw configured to be attached to the distraction plate and an actuator linkage configured to be attached to the anchor screw. The system includes an adjustor configured to be attached to the actuator linkage, wherein the adjustor rotates the actuator linkage to distract or retract the distractor plate. The position of the distractor plate may be adjusted manually by a user or may be adjusted automatically by a device comprising a motor or gearmotor that is mechanically coupled to the actuator linkage.

Periosteal distraction is a surgical technique that is traditionally used to treat bone defects by stimulating bone growth. Periosteal distraction is designed to gradually separate the periosteum from underlying bone tissue, which creates an artificial space between the bone surface and periosteum. In response, the body generates new bone tissue with no need for corticotomy. Periosteal distraction is minimally invasive when compared to traditional bone grafting techniques, and the procedure encourages the body to generate its own bone, which typically integrates more successfully than synthetic materials.

What is needed are improved systems, methods, and devices for periosteal distraction that reduce the risks of infections, reduce the risks of device complications, reduce surgery complexity, reduce the quantity of percutaneous sites prone to infection, reduce the number of external components that may be cumbersome and painful for the patient, and reduce the complexity of tasks performed after surgery. Additionally, what is needed are systems, methods, and devices for triggering regeneration of non-bone tissue types through distraction histogenesis and angiogenesis, including, for example, promoting revascularization for the healing of diabetic foot ulcers.

In view of the foregoing, described herein systems, methods, and devices for initiating tissue growth through distraction histogenesis. The distraction histogenesis techniques described herein may be utilized to treat tissue defects by raising systemic growth factor levels. One or more of the devices described herein can be coupled to many different tissues in the body, because the devices are internal and implantable.

In the following description of the disclosure, reference is made to the accompanying drawings, which form a part hereof, and in which is shown by way of illustration specific implementations in which the disclosure may be practiced. It is understood that other implementations may be utilized, and structural changes may be made without departing from the scope of the disclosure.

Before the structures, systems, methods, and compositions described herein are disclosed, it is to be understood that this disclosure is not limited to the particular structures, configurations, process steps, and materials disclosed herein as such structures, configurations, process steps, and materials may vary somewhat. It is also to be understood that the terminology employed herein is used for the purpose of describing particular embodiments only and is not intended to be limiting since the scope of the disclosure will be limited only by the appended claims and equivalents thereof.

In describing and claiming the subject matter of the disclosure, the following terminology will be used in accordance with the definitions set out below.

As used herein, the terms “comprising,” “including,” “containing,” “characterized by,” and grammatical equivalents thereof are inclusive or open-ended terms that do not exclude additional, unrecited elements or method steps.

As used herein, the phrase “consisting of” and grammatical equivalents thereof exclude any element, step, or ingredient not specified in the claim.

As used herein, the phrase “consisting essentially of” and grammatical equivalents thereof limit the scope of a claim to the specified ingredients, materials, or steps and those that do not materially affect the basic and novel characteristic or characteristics of the claimed disclosure.

Unless defined otherwise, all technical and scientific terms used herein have the same meanings as commonly understood by one of ordinary skill in the art to which this disclosure pertains and belongs.

Reference will now be made in detail to the exemplary embodiments, examples of which are illustrated in the accompanying drawings. Wherever possible, the same reference numbers are used throughout the drawings to refer to the same or like parts. It is further noted that elements disclosed with respect to particular embodiments are not restricted to only those embodiments in which they are described. For example, an element described in reference to one embodiment or figure, may be alternatively included in another embodiment or figure regardless of whether or not those elements are shown or described in another embodiment or figure. In other words, elements in the figures may be interchangeable between various embodiments disclosed herein, whether shown or not.

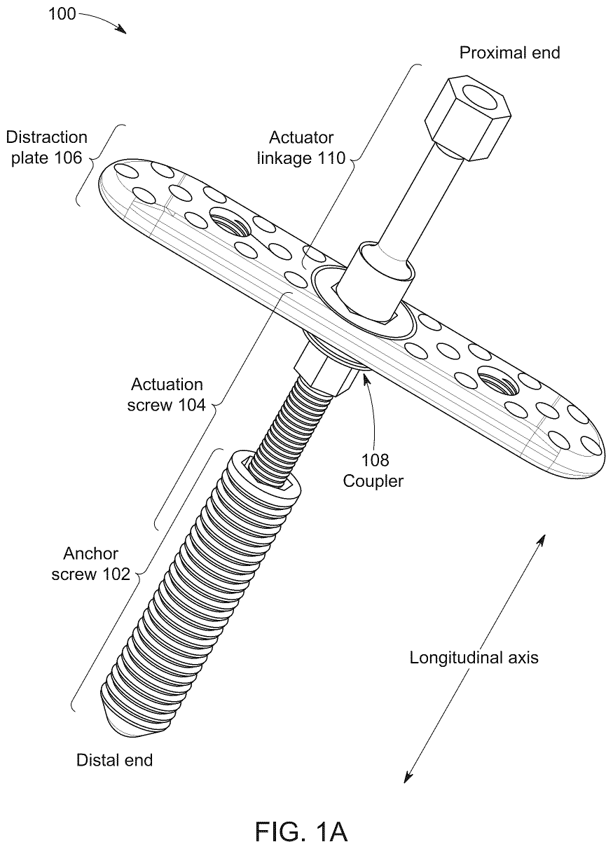

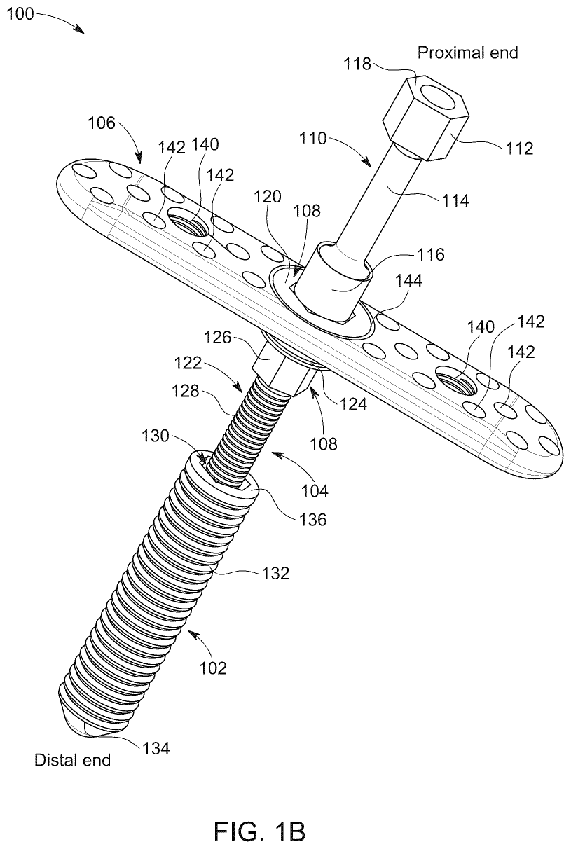

Referring now to the figures, FIGS. 1A and 1B are perspective views of a system 100 for performing a distraction histogenesis surgical procedure. The system 100 may specifically be utilized to perform a periosteal distraction surgical procedure to lift periosteum away from bone tissue and thereby trigger regeneration of bone or other soft tissues. The system 100 represents an improvement over traditional periosteal distraction systems known in the art because it reduces the quantity of screws drilled into patient bone tissue, reduces the total time required to perform the procedure, reduces the external machinery required to be managed by the patient, reduce the pain and the risk of infection related to the distraction operation, and improves the ease of distracting and retracting the distraction plate. The system 100 includes a small number of separate components and thus reduces the number of items that must be prepared and sterilized prior to the procedure.

The device 100 is configured to be implanted into a patient during a surgical procedure. After implantation, the device 100 may be distracted or retracted according to the patient's needs. In an example use-case, the device 100 is implanted in between bone tissue and a periosteum layer of a patient. The device 100 may then be coupled to an automated adjuster or manual adjuster to distract and retract the device while the device is installed within the patient.

The device 100 includes several primary components, including at least an anchor screw 102 and an actuation screw 104. The device 100 further includes a distraction plate 106 that is axially coupled to the actuation screw 104. The device 100 includes a coupler 108 that axially couples the distraction plate 106 to the actuation screw 104 while ensuring the distraction plate 106 and the actuation screw 104 are not also rotationally coupled. Thus, the coupler 108 ensures the actuation screw 104 may rotate without causing synchronous rotation of the distraction plate 106.

As shown in FIG. 1A, at least a portion of the actuation screw 104 is configured to be disposed within and encased by the anchor screw 102. The anchor screw 102 is configured to be screwed into a tissue substrate of a patient and may specifically be configured to be screwed into bone tissue. The actuation screw 104 is configured to move up and down relative to a longitudinal axis of the anchor screw 102 to adjust an overall length of the device 100. The vertical position of the actuation screw 104 relative to the anchor screw 102 may be adjusted over time to cause distraction or retraction of a second tissue of the patient.

The device 100 includes a longitudinal axis, and the actuation screw 104 is configured to move up and down along the longitudinal axis and relative to the anchor screw 102. When the actuation screw 104 is rotated in a first direction, the actuation screw 104 may be unscrewed from the anchor screw 102 and thus distracted away from the anchor screw 102. When the actuation screw 104 is rotated in a second direction, the actuation screw 104 may be screwed into the anchor screw 102 and thus retracted into the anchor screw 102. The coupler 108 ensures that the distraction plate 106 is axially coupled to the actuation screw 104 such that the distraction plate 106 also distracts away from the anchor screw 102 or retracts in toward the anchor screw 102 synchronously with the actuation screw 104. The coupler 108 further ensures that the distraction plate 106 is not rotationally coupled to the actuation screw 104, such that the distraction plate 106 may remain rotationally stationary while the actuation screw 104 is screwed into or out of the anchor screw 102. When the actuation screw 104 is distracted from the anchor screw 102 as shown in FIG. 1, the device 100 has a longer total length. When the actuation screw 104 is fully retracted into the anchor screw 102, the device 100 has its minimum total length.

The distraction plate 106 is optional depending upon the condition of the patient and the intended surgical procedure. In some cases, the distraction plate 106 is useful to gently lift or stretch a certain type of patient tissue. In other cases, the distraction plate 106 is unnecessary or may hinder the distraction of a certain type of patient tissue.

In a first exemplary use-case, the system 100 is utilized to perform a periosteal distraction procedure. In this first exemplary use-case, the anchor screw 102 is anchored into bone tissue and the distraction plate 106 is coupled to the actuation screw 104, such that distraction of the actuation screw 104 causes the distraction plate 106 to lift away from the anchor screw 102 and stretch a periosteum tissue disposed over the bone tissue.

In a second exemplary use-case, the system 100 is utilized to perform a distraction histogenesis procedure on osteotomized bone tissue. In this second exemplary use-case, the anchor screw 102 is anchored into stationary bone tissue, and a head of the actuation screw 104 is coupled to osteotomized bone tissue, such that distraction of the actuation screw 104 causes the osteotomized bone tissue to lift away from stationary bone tissue. In this second exemplary use-case, the system 100 may be utilized without the distraction plate 106.

In a third exemplary use-case, the system 100 is utilized to perform both periosteal distraction and distraction histogenesis procedure simultaneously on osteotomized bone tissue. In this third exemplary use-case, the anchor screw 102 is anchored into stationary bone tissue, and the distraction plate 106 is coupled to both the actuation screw 104 and the osteotomized bone tissue, such that distraction of the actuation screw 104 causes the distraction plate 106 to lift away from the anchor screw 102, which would cause the osteotomized bone tissue to lift away from stationary bone tissue and stretch a periosteum tissue disposed over both the bone tissue and over the distraction plate at the same time. In this third exemplary use-case, the system 100 may be utilized with the distraction plate 106.

Referring now to FIG. 1B, the anchor screw 102 of the system includes external threading 132 configured to be screwed into and securely anchored to tissue substrate of a patient. In some cases, the external threading 132 of the anchor screw 102 is optimized for screwing the anchor screw 102 into bone tissue of the patient. The anchor screw 102 additionally includes an anchor tip 134 to aid during the implantation procedure. 1 The specifications of the external threading 132 may be optimized and adjusted depending on the type of tissue the anchor screw 102 will be screwed into.

The anchor screw 102 include a sidewall 136 that defines a hollow interior. The anchor screw 102 includes a socket interface 130 disposed at a proximal end of the anchor screw 102. The socket interface 130 provides a means to directly rotate the anchor screw 102. Thus, the anchor screw 102 could be independently installed into a tissue substrate without utilizing other components of the system 100, by driving the anchor screw 102 into the tissue substrate by engaging a driver with the socket interface 130. Conversely, the anchor screw 102 could be extracted from the tissue substrate by disposing a driver within the socket interface 130 and then unscrewing the anchor screw 102 from the tissue substrate.

The anchor screw 102 includes the external threading 132 attached to an exterior surface of the sidewall 136, and additionally includes internal threading (not visible in FIG. 1B) attached to an interior surface of the sidewall 136. The internal threading corresponds with external threading 128 of an outer screw shaft 122 of the actuation screw 104 such that the actuation screw 104 may be securely and releasably screwed into the hollow interior of the anchor screw 102. Thus, the actuation screw 104 may be screwed into the hollow interior of the anchor screw 102, and the actuation screw 104 may likewise be screwed out of the hollow interior of the anchor screw 102.

When the system 100 is implanted into a patient, a surgeon may first place the distraction plate 106 in the planned location such that the distraction plate rests on a first patient tissue. In some cases, the distraction plate 106 may be disposed in between the first patient tissue and a second patient tissue that is located external relative to the first patient tissue. When the distraction plate 106 is disposed in the desired location, the surgeon may utilize one or more of the holes disposed through the distraction plate 106 as a guide to drill a pilot hole into the first patient tissue and/or anchor the distraction plate 106 to the first patient tissue. After the surgeon has drilled the one or more pilot holes, the surgeon may then install the anchor screw 102 into the first patient tissue. The actuation screw 104 may be disposed within the anchor screw 102 such that the surgeon may simultaneously drive each of the anchor screw 102 and the actuation screw 104 through the central hole of the distraction plate 106 and into the pilot hole disposed within the first patient tissue. When the anchor screw 102 is fully installed, the surgeon may then distract or retract the actuation screw 104 and thereby cause the actuation screw 104 to distract away from the anchor screw 102 (as shown in FIG. 1B) or retract toward the anchor screw 102. The system 100 is designed such that the distraction plate 106 will remain rotationally stationary when the actuation screw 104 is distracted and retracted. Thus, a distraction rotation of the actuation screw 104 causes the distraction plate 106 to lift away from the anchor screw 102 as shown in FIG. 1B but does not cause the distraction plate 106 to synchronously rotate with the actuation screw 104.

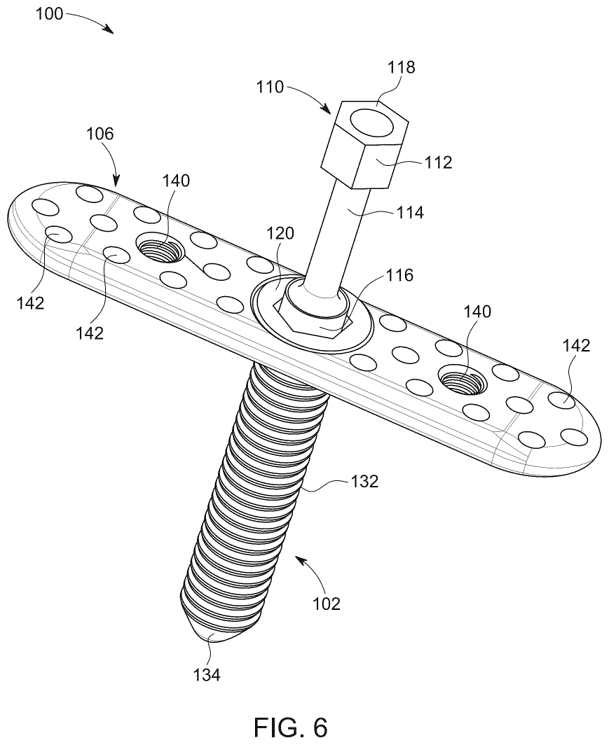

The anchor screw 102 is intended to be securely implanted within a tissue substrate, such as bone tissue. Thus, the anchor screw 102 remains stationary when the system 100 is distracted and retracted. The actuation screw 104 is configured to be fully disposed within the anchor screw 102 when the system 100 is in a fully retracted state (see, e.g., FIG. 6). The actuation screw 104 is configured to be partially exposed, and not fully disposed within the anchor screw 102, when the system 100 is in a distracted state as shown in FIG. 1B.

The system 100 includes the coupler 108 that is configured to axially couple the distraction plate 106 to the actuation screw 104 while ensuring the distraction plate 106 is not also rotationally coupled to the actuation screw 104. The coupler 108 includes an anchoring interface 120 and a rotational interface 126. The rotational interface 126 is attached to the anchoring interface 120, and the rotational interface 126 is located distal relative to the anchoring interface 120. The anchoring interface of the coupler 108 includes external threading 124 that corresponds with internal threading of a central threaded hole 144 of the distraction plate 106. The external threading 124 of the anchoring interface 120 thus enables the coupler 108 to be screwed into and secured to the distraction plate 106.

The rotational interface 126 includes a driver head configured to interface with the corresponding socket head 130 of the anchor screw 102. The rotational interface 126 may include a polygonal cross-sectional geometry that matches the polygonal cross-sectional geometry of the socket interface 130 of the anchor screw 102. In the example illustrated in FIGS. 1A-1B, the rotational interface 126 includes a hexagonal cross-sectional geometry, and may thus be referred to as a hex driver. The exemplary socket interface 130 illustrated in FIGS. 1A-1B corresponds with the rotational interface 126 and thus includes a hex socket. It should be appreciated that the rotational interface 126 and the socket interface 130 may include various numbers of sides as deemed appropriate, including, for example, a quadrilateral, pentagonal, hexagonal, octagonal, and so forth, polygonal cross-sectional geometry.

The coupler 108 comprises a hollow interior and a through hole, such that the coupler 108 is configured to receive the actuation screw 104. In the orientation illustrated in FIG. 1B, the actuation screw 104 extends up through the coupler 108 and then interfaces with the actuator linkage 110. The coupler 108 comprises a smooth hollow interior that enables the actuation screw 104 to freely rotate within the coupler 108. Thus, the coupler 108 enables the actuation screw 104 to rotate out of and into the anchor screw 102 without causing synchronous rotation of the distraction plate 106.

The coupler 108 enables the distraction plate 106 to be axially coupled to the actuation screw 104 such that distraction plate 106 moves vertically along the longitudinal axis of the system 100 while remaining rotationally stationary when the actuation screw 104 is screwed into and out of the anchor screw 102.

The distraction plate 106 includes a central threaded hole 144, one or more peripheral threaded holes 140, and a plurality of holes 142 disposed therethrough. The holes 142 may allow fluids, cells, molecules, and other components to pass between the upper and lower surfaces of the distraction plate 106, and this may improve patient outcomes from the distraction histogenesis and angiogenesis procedure. Each of the central threaded hole 144 and the one or more peripheral threaded holes 140 may include internal threading. The internal threading of the central threaded hole 144 corresponds with the external threading 124 of the anchoring interface 120 of the coupler 108. The internal threading of the one or more peripheral threaded holes 140 may corresponds with screws intended for anchoring the distraction plate 106 to patient tissue and may specifically be utilized to anchor the distraction plate 106 to osteotomized bone tissue.

The actuator linkage 110 is releasably coupled to a screw head (not visible in FIG. 1B) of the actuation screw 104. The actuator linkage 110 may thus be utilized to drive rotation of the actuation screw 104. The actuator linkage 110 serves as a bridge between the actuation screw 104 and an exterior of the patient's body. Thus, if the system 100 is utilized to perform a periosteal distraction procedure, the actuator linkage 110 percutaneously extends through the periosteum and other tissue layers located external to the periosteum, such as fat, skin, and other tissues.

The actuator linkage 110 includes an actuation screw coupling head 116, a shaft 114, and rotator coupling head 118. The actuation screw coupling head 116 has an internal recess socket feature which is configured to interface with a corresponding screw head (not visible in FIG. 1B) on the actuation screw 104, which serves as a torque drive component of the actuation screw 104. The rotator coupling head 118 is configured to interface with the internal socket feature of the interface 120 of the coupler 108. Through this interface coupling, the rotation of the coupling head 118 can rotate the coupler 108. The rotator coupling head 118 is also configured to interface with a corresponding component utilized to rotate the actuator linkage 110. This corresponding component may include, for example, a knob enabling a user to manually rotate the actuator linkage 110, or a component for automatically rotating the actuator linkage 110 with an automated fixator adjustor. The rotator coupling head 118 may include a plurality of sides 112 forming a polygonal cross-sectional geometry, which may be utilized to rotate the actuator linkage more easily 108.

In some cases, a knob (not shown in FIGS. 1A-1B) may be releasably coupled to the actuator linkage 110 to enable a user to easily rotate the actuator linkage 110, and thereby also rotate the actuation screw 104. In other cases, a component of an automated fixator adjustor may be releasably coupled to the actuator linkage 110 to automatically rotate the actuator linkage 110 according to a treatment schedule.

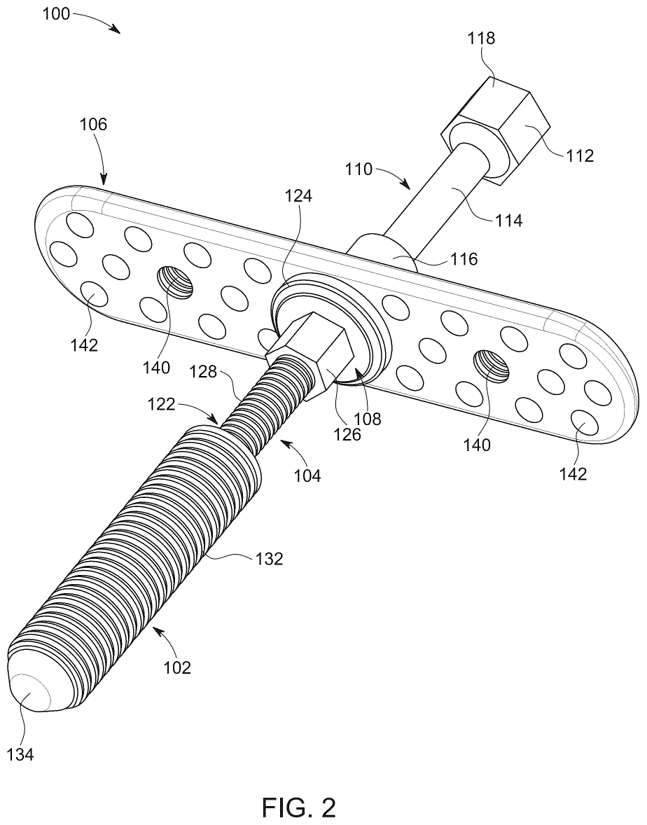

FIG. 2 is an underside perspective view of the system 100 for performing a distraction histogenesis surgical procedure. Like in FIGS. 1A-1B, FIG. 2 also illustrates wherein the actuation screw 104 is partially distracted (i.e., screwed out of) the anchor screw 102, and thus, the distraction plate 106 is lifted away from the anchor screw 102.

As shown in FIG. 2, the external threading 124 of the coupler 108 may be screwed into a threaded hole of the distraction plate 106 to securely couple the coupler 108 to the distraction plate 106. Further as shown in FIG. 2, the rotational interface 126 of the coupler 108 may be directly attached to the outer screw shaft 122 of the actuation screw 104.

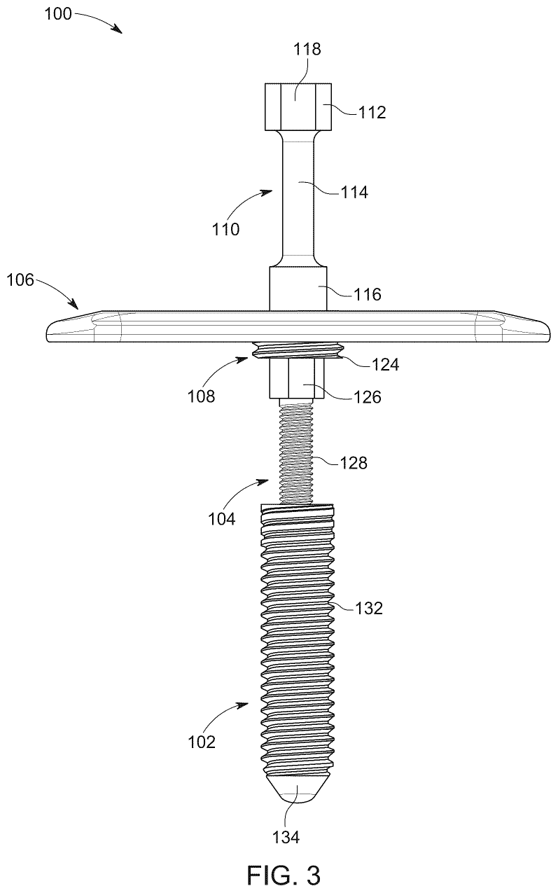

FIG. 3 is a straight-on side view of the system 100 for performing a distraction histogenesis surgical procedure. Like in FIGS. 1A-1B and 2, FIG. 3 also illustrates wherein the actuation screw 104 is partially distracted (i.e., screwed out of) out of the anchor screw 102, and thus, the distraction plate 106 is lifted away from the anchor screw 102.

When the system 100 is implanted in a patient, the anchor screw 102 may be screwed into a first tissue of the patient while the actuation screw 104 is fully screwed into the anchor screw 102. If the distraction plate 106 is being utilized, then a surgeon may first place the distraction plate 106 in a desired location on the patient, and then screw the anchor screw 102 through a hole disposed through the distraction plate 106. The actuator linkage 110 may then be coupled to the actuation screw 104 through the hole disposed through the distraction plate 106. Typically, a surgeon will close up the patient skin layer such that the actuator linkage 110 is the only component located external to the patient. The actuator linkage 110 thus serves as a bridge through one or more layers of patient tissue and enables a practitioner to distract or retract the actuation screw 104 without reopening the patient's skin layer. When the actuation screw 104 is distracted (i.e., lifted or screwed out of the anchor screw 102, as shown in FIG. 3), the actuation screw 104 and distraction plate 106 will lift a second patient tissue layer away from the first patient tissue layer. In practice, the anchor screw 102 may be screwed into bone tissue, and the actuation screw 104 and distraction plate 106 may lift a periosteum up and away from the bone tissue.

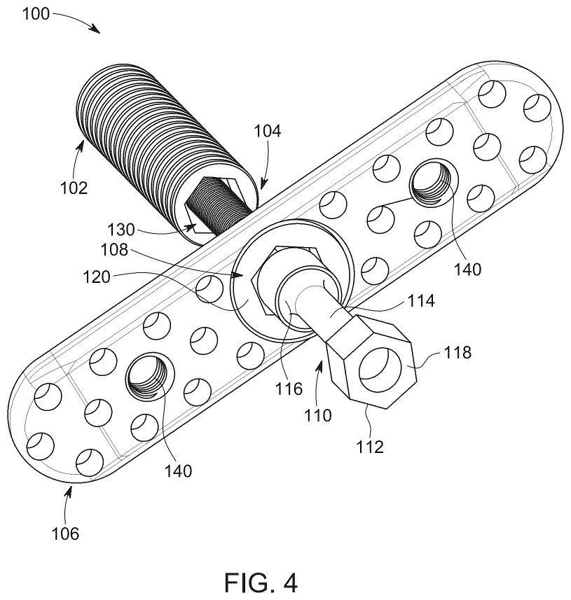

FIG. 4 is an overhead top-down perspective view of the system 100 for performing a distraction histogenesis surgical procedure. Like in FIGS. 1A-1B and 2-3, FIG. 4 also illustrates wherein the actuation screw 104 is partially distracted out of (i.e., screwed out of) the anchor screw 102, and thus, the distraction plate 106 is lifted away from the anchor screw 102.

As shown in FIG. 4, the rotator coupling head 118 of the actuator linkage 110 may comprise a plurality of sides 112 such that the rotator coupling head 118 comprises a polygonal cross-sectional geometry. The exemplary rotator coupling head 118 illustrated in FIG. 4 includes a hexagonal cross-sectional geometry, but it should be appreciated that the rotator coupling head 118 may comprise other cross-sectional geometries without departing from the scope of the disclosure. The polygonal cross-sectional geometry of the rotator coupling head 118 enables a user or automated fixator adjustor to achieve sufficient torque to rotate the actuator linkage 110 and the actuation screw 104.

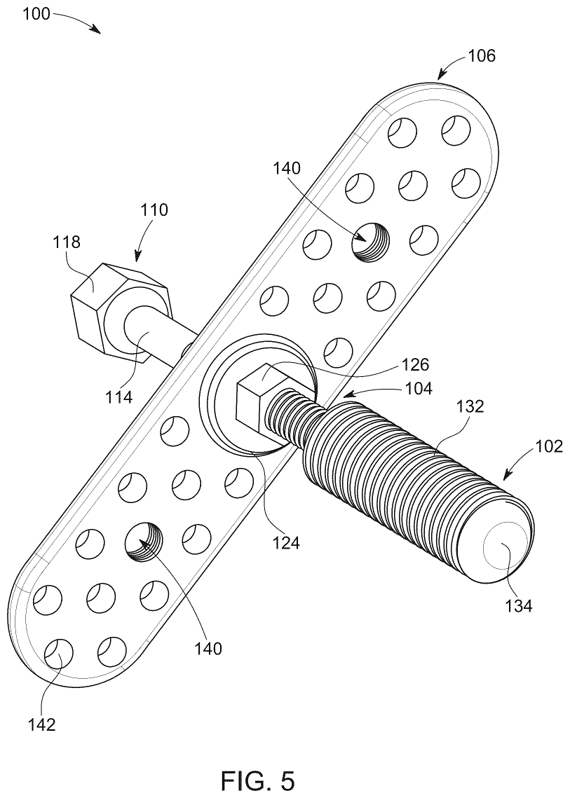

FIG. 5 is an underside perspective view of the system 100 for performing a distraction histogenesis surgical procedure. Like in FIGS. 1A-1B and 2-4, FIG. 5 also illustrates wherein the actuation screw 104 is partially distracted out of (i.e., screwed out of) the anchor screw 102, and thus, the distraction plate 106 is lifted away from the anchor screw 102.

FIG. 6 is a perspective view of the system 100 for performing a distraction histogenesis procedure, wherein the actuation screw (see 104, not visible in FIG. 6) is fully retracted into the anchor screw 102 (i.e., fully screwed into the anchor screw 102).

In practice, the system 100 may be disposed like in FIG. 6 when the system 100 is first installed into a patient and/or when the system 100 is about to be removed from the patient. After installation, a user or automated fixator adjustor may rotate the actuator linkage 110 to thereby rotate the actuation screw (see 104 as first illustrated in FIG. 1). When the actuation screw 104 is unscrewed from the anchor screw 102, the actuation screw 104 will lift away from the anchor screw 102. If the optional distraction plate 106 is being utilized, then the distraction plate 106 will also lift away from the anchor screw 102. The user or automated fixator adjustor may also rotate the actuator linkage 110 in the opposite direction to retract the actuation screw (i.e., screw the actuation screw into the anchor screw).

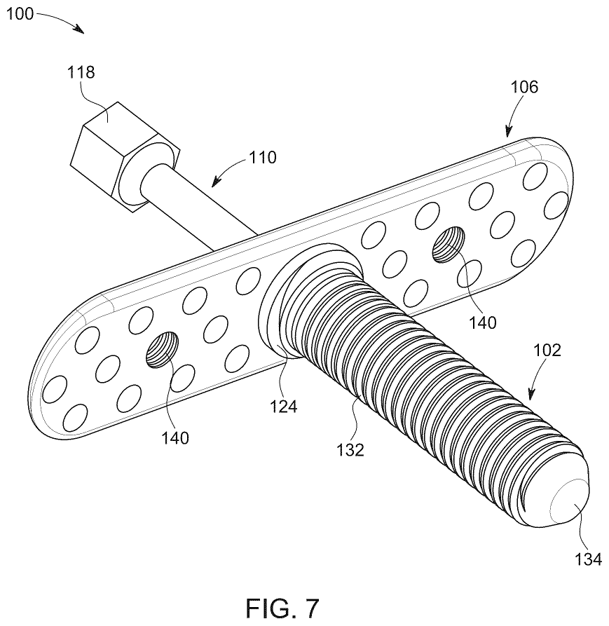

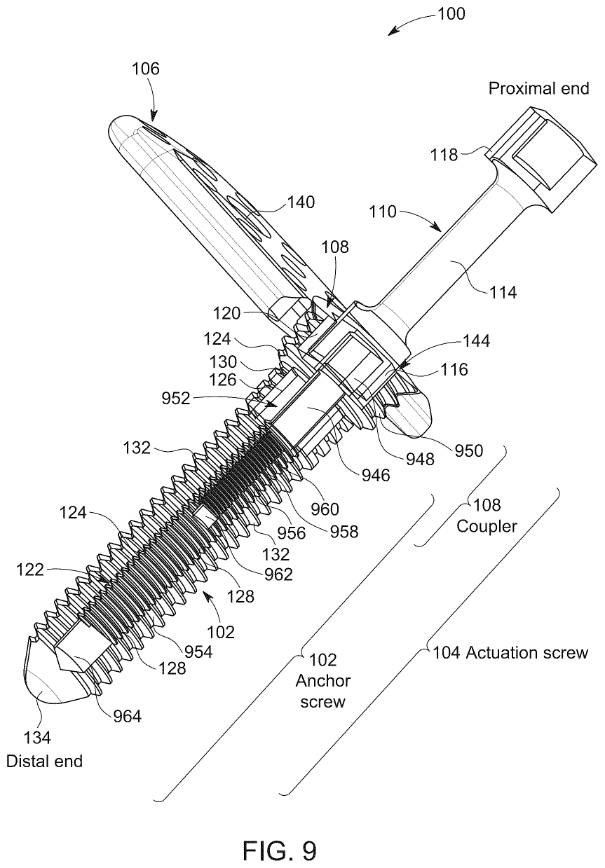

FIG. 7 is a perspective view of the system 100 for performing a distraction histogenesis procedure, wherein the actuation screw is fully retracted into the anchor screw 102. As shown in FIG. 7, the screw head (see 948 at FIG. 9) of the actuation screw 104 may be disposed within the hollow interior defined by the sidewall of the anchor screw 102. Thus, the screw head (see 948) is not visible when the actuation screw 104 is fully retracted into the anchor screw. However, the sidewall of the anchor screw 102 is designed such that the actuation screw coupling head 116 of the actuator linkage 110 may still access the screw head (see 948) to rotate the actuation screw 104.

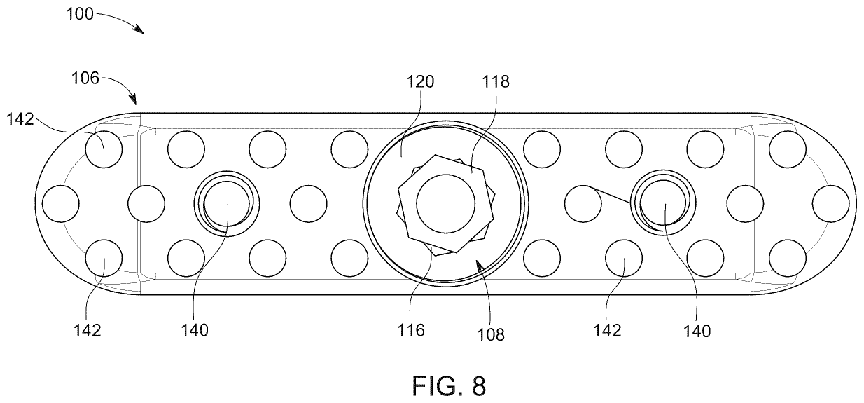

FIG. 8 is a straight-on aerial top-down view of the system 100 for performing a distraction histogenesis procedure. The aerial view of FIG. 8 provides further viewing of the various holes disposed through the distraction plate 106, including the peripheral threaded holes 140 and the holes 142.

FIG. 9 is a perspective cross-sectional view of the system 100 for performing a distraction histogenesis procedure, wherein the actuation screw is fully retracted into the anchor screw 102. As shown in the cross-sectional view, the actuation screw 104 is disposed within a hollow interior space defined by the anchor screw 102.

The cross-sectional view of FIG. 9 enables viewing of the full length of each of the anchor screw 102, the actuation screw 104, and the coupler 108. As shown in FIG. 9, the anchor tip 134 of the anchor screw 102 serves as the distal-most point of the device 100. The actuation screw 104 is disposed within the anchor screw 102 and is further disposed through the coupler 108. The coupler 108 extends through the distraction plate 106 and partially down into the anchor screw 102 when the device is in a fully retracted position as shown in FIG. 9.

The cross-sectional view of FIG. 9 further enables viewing of various components of the actuation screw 104. The actuation screw 104 may include each of the outer screw shaft 122 and an inner screw shaft 960, wherein the inner screw shaft 960 is configured to be disposed at least partially within the outer screw shaft 122.

The outer screw shaft 122 comprises the external threading 124. The proximal end of the outer screw shaft 122 is located at a distal end of the coupler 108. The distal end of the outer screw shaft 122 is formed by an outer actuation point 964. The outer actuation point 964 is configured to be disposed within the anchor screw 102 and may nearly reach the distal-most end of the anchor screw 102. The entirety of the outer screw shaft 122 is located distal relative to the coupler 108 and the distraction plate 106.

A diameter of the outer screw shaft 122 is greater than an inner diameter of the through-holed defined by the coupler 108. The diameter of the outer screw shaft 122 may additionally be greater than an inner diameter of the central threaded hole of the distraction plate 106. Thus, the outer screw shaft 122 may not be disposed within the through-hole defined by the coupler 108, and the outer screw shaft 122 is thus maintained in a position that is distal relative to the coupler 108 and the distraction plate 106.

The external threading 124 of the outer screw shaft 122 is configured to interface with internal threading 954 attached to an interior surface of the sidewall 136 of the anchor screw 102. At least a portion of the outer screw shaft 122 includes a sidewall that defines a hollow interior, and an interior surface of the outer screw shaft 122 may include internal threading 956. The internal threading 956 is configured to interface with corresponding external threading 958 of the inner screw shaft 960 of the actuation screw.

The inner screw shaft 960 is configured to be disposed within at least a portion of the outer screw shaft 122. The proximal end of the inner screw shaft 960 comprises a screw head 948 that may be located within a portion of the coupler 108 and/or the distraction plate 106. The distal end of the inner screw shaft 960 comprises an inner actuation point 962 that is disposed within the hollow interior formed by the outer screw shaft 122. The inner screw shaft 960 is configured to be disposed through the through-hole of the coupler 108, and thus, in combination with the coupler 108, the inner screw shaft 960 enables the actuation screw 104 to be axially coupled to the distraction plate 106 without also being rotationally coupled to the distraction plate 106.

The inner screw shaft 960 includes a threaded portion that includes the external threading 958. The inner screw shaft 960 may additionally include a smooth portion 946. The smooth portion 946 is configured to be disposed within a hollow interior 952 defined by the rotational interface 126 of the coupler 108. Thus, the smooth portion 946 may rotate freely within the rotational interface 126 of the coupler 108.

The screw head 948 of the inner screw shaft 960 of the actuation screw 104 is configured to interface with the actuation screw coupling head 116 of the actuator linkage 110. The screw head 948 is surrounded by the hollow interior defined by the anchoring interface 120 of the coupler 108. The screw coupling head 116 may be disposed within the hollow interior space located in between the inner surface of the anchoring interface 120 of the coupler 108, and the outer surface of the screw head 948 of the actuation screw 104. When the screw coupling head 116 is located within that hollow interior space, the screw coupling head 116 may form an interference fit with the screw head 948 and then drive rotation of the inner screw shaft 960 of the actuation screw 104. This may likewise drive rotation of the actuation screw 104 without driving synchronous rotation of the coupler 108 or the distraction plate 106.

The rotational interface 126 of the coupler 108 includes a polygonal cross-sectional exterior geometry that enables the rotational interface 126 to form a torque coupling with the corresponding polygonal cross-sectional interior geometry of the socket interface 130 of the anchor screw 102. This enables a user to simultaneously implant the actuation screw 104 and the anchor screw 102 into a patient as a single unit.

Further as shown in the cross-sectional view, the central threaded hole 144 of the distraction plate includes internal threading 950 that corresponds with the external threading 124 of the coupler 108. This enables the coupler 108 to be securely screwed into the distraction plate 106. Further as shown in the cross-sectional view, the anchor screw 102 includes internal threading 954 that corresponds with the external threading 128 of the actuation screw 104.

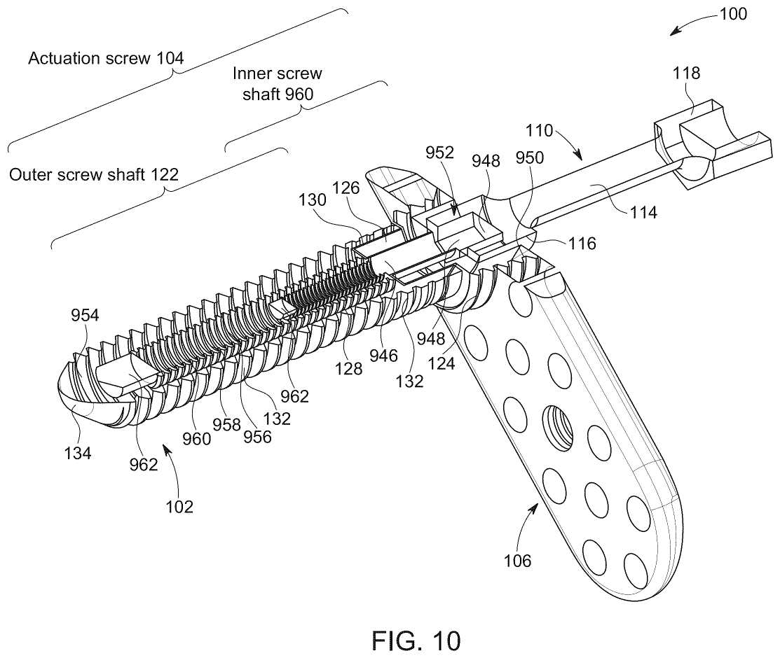

FIG. 10 is a perspective cross-sectional view of the system 100 for performing a distraction histogenesis procedure, wherein the actuation screw is fully retracted into the anchor screw 102. The cross-sectional view illustrated in FIG. 10 shows that the screw head 948 of the actuation screw 104 may have an external cross-sectional polygonal geometry that corresponds with an internal cross-sectional polygonal geometry of the actuation screw coupling head 116 of the actuator linkage 110. This ensures the actuator linkage 110 may have sufficient torque to rotate the actuator screw 104.

The brackets in FIG. 10 further illustrate how the combination of the outer screw shaft 122 and the inner screw shaft 960 make up the actuation screw 104. As shown, the inner screw shaft 960 is partially disposed within the outer screw shaft 122, and additionally passes through the coupler 108 and terminates within the central threaded hole of the distraction plate 106.

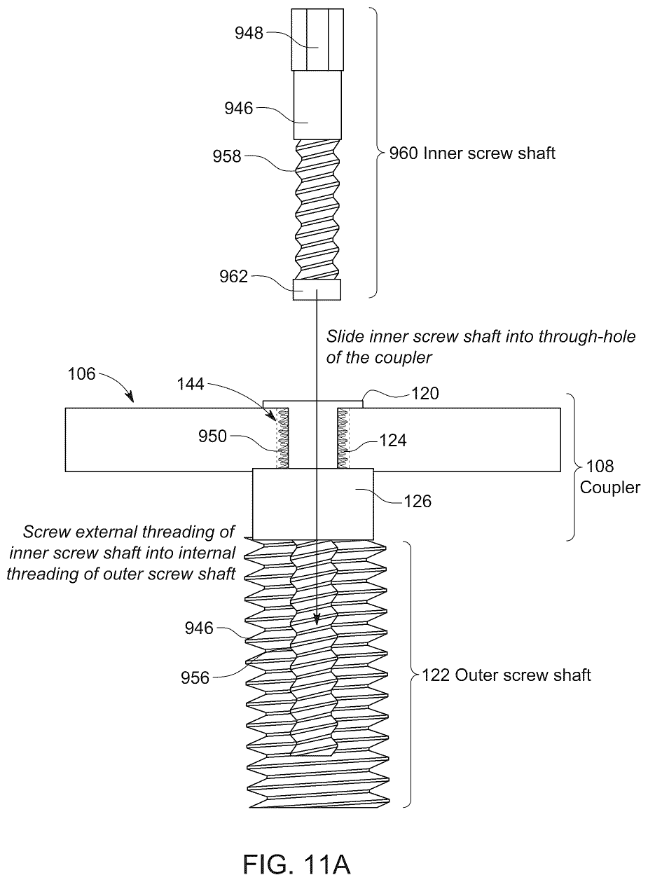

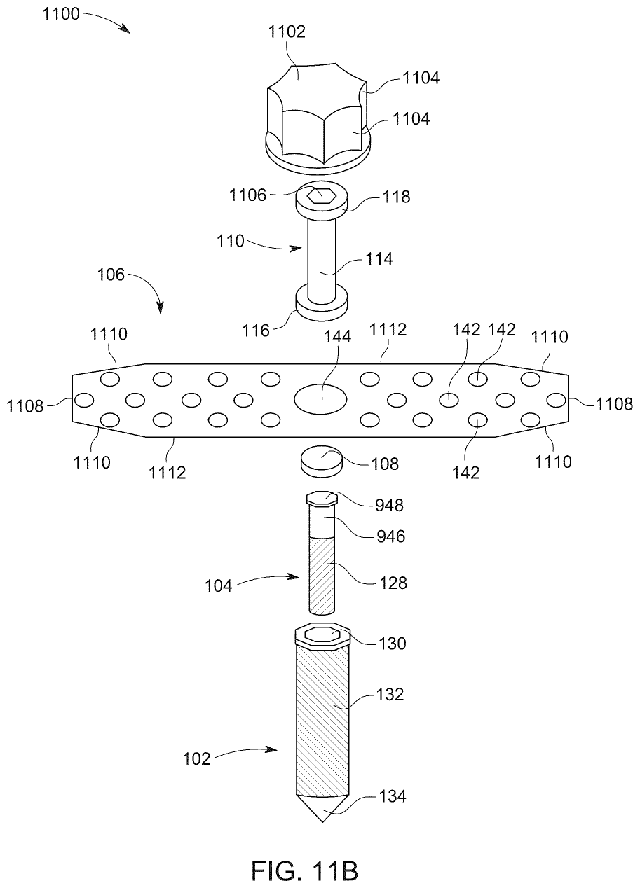

FIG. 11A is a schematic illustration of an exploded straight-on side view of an anti-rotation assembly, which is a component of the device 100 described herein. The anti-rotation assembly includes components of the actuation screw 104 (including the inner screw shaft 960 and the outer screw shaft 122), the coupler 108, and the distraction plate 106. The anti-rotation assembly enables the distraction plate 106 to be axially coupled to the actuation screw 104 without also being rotationally coupled to the actuation screw 104.

The anti-rotation assembly includes the actuation screw 104, which includes the inner screw shaft 960 and the outer screw shaft 122. The outer screw shaft 122 comprises a diameter that is greater than a diameter of the rotational interface 126 of the coupler 108, and this prevents the outer screw shaft 122 from sliding up into the coupler 108 or the distraction plate 106. The inner screw shaft 960 comprises a diameter that is smaller than the diameter of the central threaded hole 144 of the distraction plate 106. The diameter of the inner screw shaft 960 is also smaller than the diameter of a through-hole extending through the entirety of the coupler 108, including the anchoring interface 120 and the rotational interface 126.

During assembly or installation, the inner screw shaft 960 is slid through the through-hole defined by the sidewall of the coupler 108. The inner screw shaft 960 is then securely coupled to the outer screw shaft 122 by screwing the external threading 958 of the inner screw shaft 960 into the internal threading 956 disposed within the hollow interior of the outer screw shaft 122. The inner screw shaft 960 will be fully installed within the outer screw shaft 122 when the screw head 948 is located approximately within the central threaded hole 144 of the distraction plate 106 and/or when the smooth portion 946 of the inner screw shaft 960 is aligned with the rotational interface 126 of the coupler 108. The outer screw shaft 122 may include stopper that prevents the inner screw shaft 960 from being screw in any deeper than desired.

As shown in FIG. 11A, the rotational interface 126 of the coupler 108 comprises a diameter that is greater than a diameter of the smooth portion 946 of the inner screw shaft 960. This aids in enabling the inner screw shaft 960 to rotate freely within the coupler 108. This consequently ensures the inner screw shaft 960 may rotate freely within the distraction plate 106, and this enables the distraction plate 106 to remain rotationally stationary even when the actuation screw 104 is screwed into or screwed out of the anchor screw 102.

The external threading 958 of the inner screw shaft may be tightly engaged with the internal threading 956 of the outer screw shaft 122 to prevent these two components of the actuation screw 104 from becoming separated. In some cases, the inner screw shaft 960 may be glued into the outer screw shaft 122 to ensure a tight and semi-permanent installation. This may help ensure that a loosening rotation of the actuation screw 104 to remove the actuation screw from the anchor screw 102 does not also loosen the inner screw shaft 960 from the outer screw shaft 122.

FIG. 11B is a schematic illustration of an exploded perspective view of a system 1100 for performing a distraction histogenesis procedure. The system 1100 includes the components of the system 100 first described in connection with FIGS. 1A-1B. The system 1100 additionally includes a knob 1102 for enabling easy rotation of the actuator linkage 110.

The knob 1102 may include a plurality of grooves 1104 to enable a user to rotate the knob 1102 more easily clockwise or counterclockwise. The knob 1102 may comprise various implementations without departing from the scope of the disclosure. For example, the knob 1102 may comprise a smooth cylindrical geometry without any grooves 1104. The knob 1102 may comprise a quadrilateral, pentagonal, hexagonal, octagonal, or other cross-sectional geometry to provide straight sides enabling a user to rotate the knob 1102 more easily. The knob 1102 may be constructed of any suitable rigid material, including, for example, a metallic material, a polycarbonate material, and so forth. The knob 1102 may be constructed of a material that may be easily cleaned to prevent the buildup or absorption of fluids within the knob 1102. The knob 1102 may also comprise a ball plunger which would provide tactile feedback to users when they rotate the knob.

The actuator linkage 110 may comprise a polygonal blind hole 1106 disposed within a surface of the rotator coupling head 118. The polygonal blind hole 1106 enables the actuator linkage 110 to interface with corresponding components disposed within a bottom surface of the knob 1102 or a corresponding component within an automated fixator adjustor. The blind hole 1106 may include polygonal cross-sectional geometries to provide the necessary torque to enable the knob 1102 or automated fixator adjustor to rotate the actuator linkage 110, and thereby enable the actuator linkage 110 to rotate the actuation screw 104.

The actuator linkage 110 may comprise male or female components for forming a coupling with the knob 1102 and the actuation screw 104. In some cases, the actuator linkage 110 may comprise a male component on the rotator coupling head 118 and a female component on the actuation screw coupling head 116, or vice versa.

The distraction plate 106 comprises the plurality of holes 142 disposed therethrough, and additionally includes the central hole 144 disposed through a center of the distraction plate 106. The central hole 144 may comprise internal threading such that one or more of a drill sleeve (not pictured) or the coupler 108 may be threaded through the central hole 144.

The distraction plate 106 may comprise various geometries without departing from the scope of the disclosure. The distraction plate 106 may comprise an irregular octagonal geometry as shown in FIG. 11A, wherein the distraction plate 106 includes two long sides 1112 oriented parallel to one another, two short sides 1108 oriented parallel to one another and perpendicular to the two long sides 1112, and four chamfered sides 1110. Each of the four chamfered sides 1110 is disposed in between one long side 1112 and one short side 1108 as shown in FIG. 11A. The two long sides 1112 may have substantially equivalent lengths, the short sides 1108 may have substantially equivalent lengths, and the four chamfered sides 1110 may have substantially equivalent lengths.

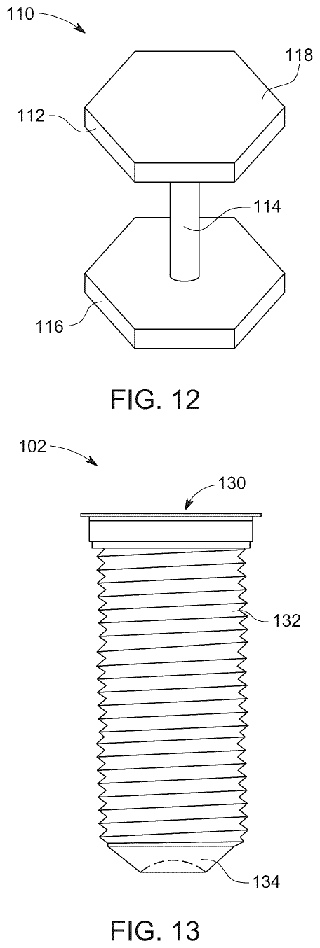

FIG. 12 is a schematic illustration of a perspective of the actuator linkage 110. As shown, the actuator linkage 110 comprises the rotator coupling head 118, the shaft 114, and the actuation screw coupling head 116. Each of the rotator coupling head 118 and the actuation screw coupling head 116 may comprise a polygonal cross-sectional geometry to enable the actuator linkage 110 to provide sufficient torque such that the knob (see 1102) or automated fixator adjustor can rotate the anchor screw 108.

The polygonal cross-sectional geometries of the rotator coupling head 118 and the actuation screw coupling head 116 may vary without departing from the scope of the disclosure. One or more of the rotator coupling head 118 or the actuation screw coupling head 116 may comprise a hexagonal cross-sectional geometry as shown in FIG. 11A. However, one or more of the rotator coupling head 118 or the actuation screw coupling head 116 may alternatively comprise one or more of a triangular, quadrilateral, pentagonal, heptagonal, octagonal, or n-sided geometry, depending on the implementation.

FIG. 13 is a schematic illustration of a straight-on side view of the anchor screw 102. As shown, the anchor screw 102 comprises a socket interface 130, external threading 132, and an anchor tip 134. The external threading 132 and the design of the anchor tip 134 may be adjusted and optimized depending on the intended use-case based on factors such as the type of bone being operated upon, the patient's condition, the patient's age, and so forth.

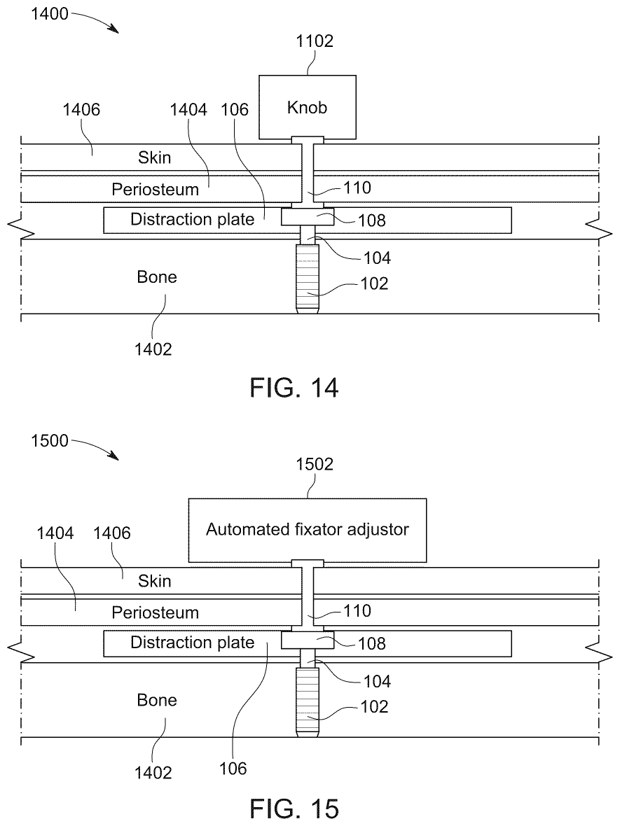

FIG. 14 is a schematic block diagram of a system 1400 for manual distraction and retraction, and specifically illustrates wherein system components are installed in a patient. The system 1400 includes the distraction plate 106 disposed in between the bone 1402 and the periosteum 1404 of the patient. The system 1400 further includes the anchor screw 102 drilled into the bone 1402 of the patient, and the actuation screw 104 disposed within the anchor screw 102. As shown, the skin 1406 is located external to the periosteum 1404, but it should be appreciated that additional tissues and biological components may be disposed in between the periosteum 1404 and the skin 1406. The actuator linkage 110 is coupled to the actuation screw 104 and the knob 1102, and the shaft of the actuator linkage 110 is disposed through patient tissue. The knob 1102 is located external to the patient, such that a patient or practitioner may rotate the knob to rotate the actuation screw 104 and thereby distraction or retract the distraction plate 106.

FIG. 15 is a schematic block diagram of a system 1500 for automated distraction and retraction through mechanical coupling, and specifically illustrates wherein system components are installed in a patient. The system 1500 includes the distraction plate 106 disposed in between the bone 1402 and the periosteum 1404 of the patient. The system 1500 further includes the anchor screw 102 drilled into the bone 1402 of the patient, and the actuation screw 104 disposed within the anchor screw 102. As shown, the skin 1406 is located external to the periosteum 1404, but it should be appreciated that additional tissues and biological components may be disposed in between the periosteum 1404 and the skin 1406. The actuator linkage 110 is attached to the actuation screw 104 and an automated fixator adjustor 1502, and the shaft of the actuator linkage 110 is disposed through patient tissue. The automated fixator adjustor 1502 is located external to the patient, such that a patient or practitioner may cause a motor within the automated fixator adjustor 1502 to automatically rotate the actuation screw 104.

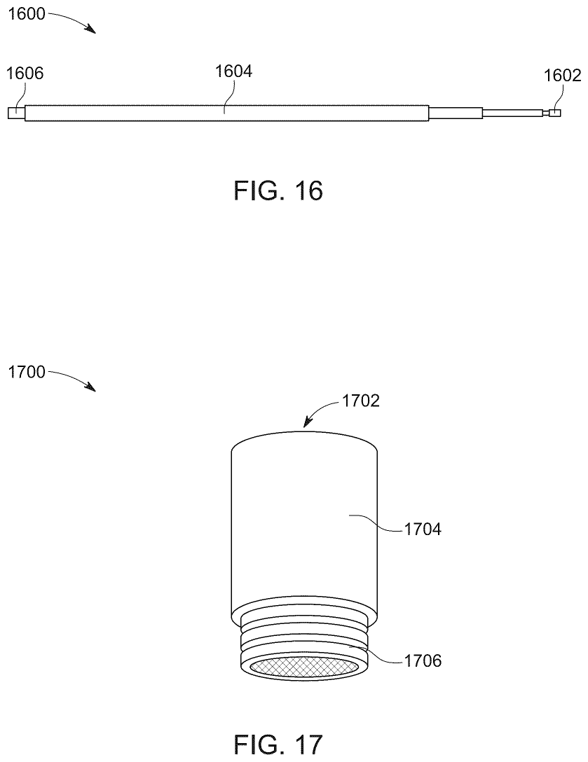

FIG. 16 is a schematic illustration of a straight-on wide view of a cannulated driver 1600 to be used in connection with the components of the systems described herein. The cannulated driver 1600 comprises a distal end 1602, a shaft 1604, and a proximal end 1606. The distal end 1602 comprises a polygonal cross-sectional geometry configured to interface with the socket interface (see 130) of the anchor screw (see 102). This provides sufficient torque such that the cannulated driver 1600 may be utilized to screw the anchor screw (see 102) into the bone of a patient and into the distraction plate 106 simultaneously. The proximal end 1606 is configured to interface with another device such as a screwdriver handle, drill, or other device.

The socket interface (see 130) of the anchor screw (see 102) includes a polygonal cross-sectional geometry on its bottom side that is configured to mate with the shaft of the anchor screw (see 102). This enables torque to be transmitted from the cannulated driver 1600 to the socket interface (see 130) and then to the shaft of the anchor screw (see 102) to drive the anchor screw (see 102) into patient tissue.

FIG. 17 is a schematic illustration of a perspective view of drill sleeve 1700 to be used in connection with the components of the system 100 described herein. The drill sleeve 1700 is configured to be utilized by a healthcare provider to enable the provider to drill a pilot hole into the patient bone. The drill sleeve 1700 includes a shaft 1704 that comprises a hollow interior 1702. The drill sleeve 1700 further comprises threading 1706 that corresponds with the internal threading of the central hole of the distraction plate (see 106). Thus, the drill sleeve 1700 may be securely screwed into the distraction plate (see 106), and then a provider may dispose a drill bit through the hollow interior 1702 of the shaft 1704. This guides the location of the drill bit and prevents the provider from drilling into the patient bone at an undesired location and also makes sure the anchor screw to be inserted will be perpendicular to the distraction plate.

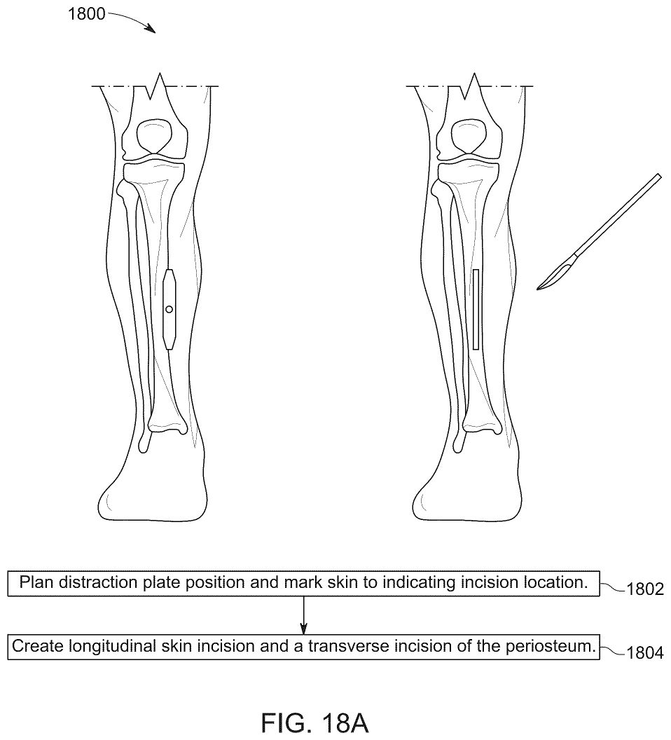

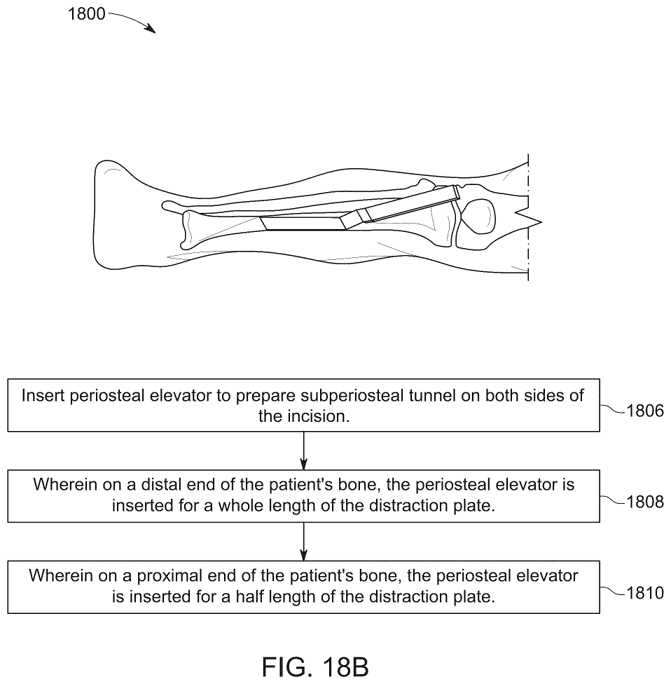

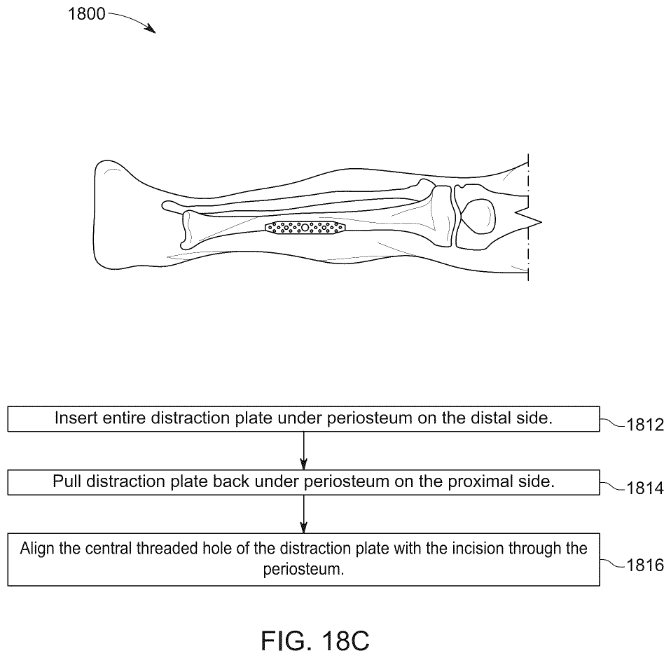

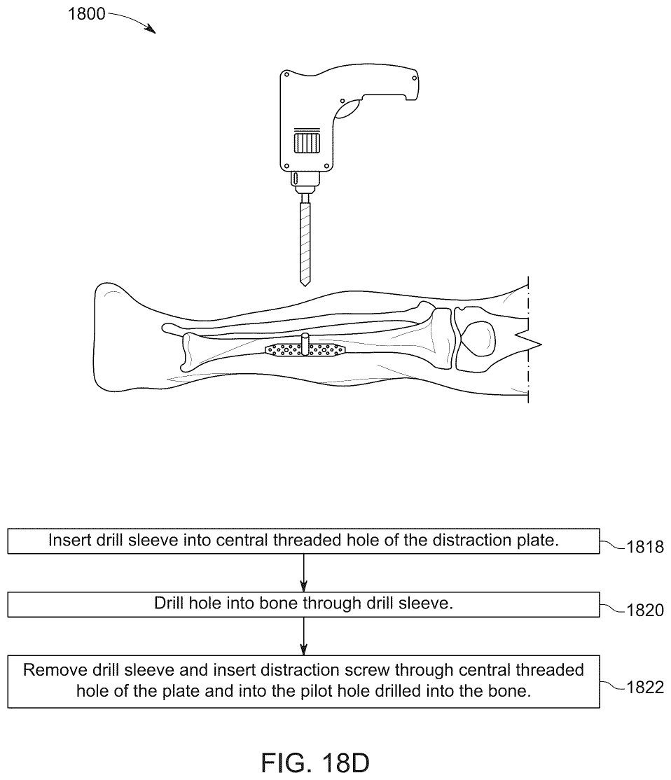

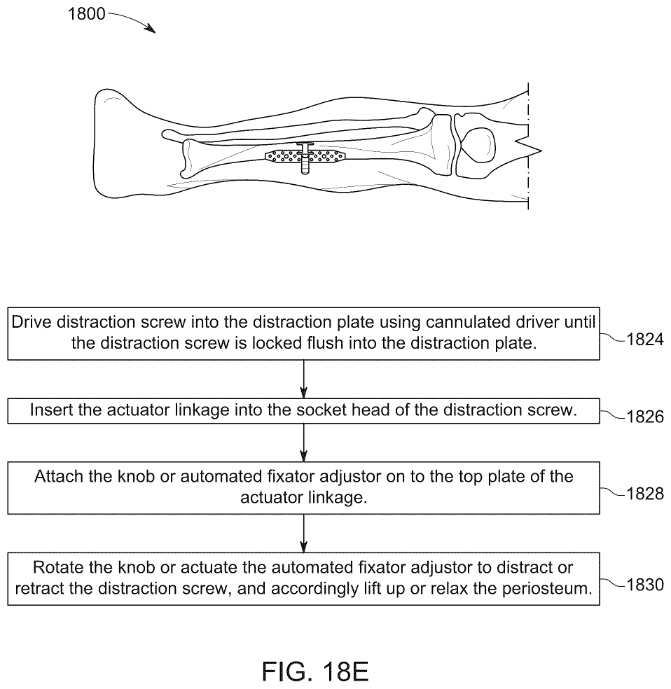

FIGS. 18A-18E are schematic block diagrams of a method 1800 for performing a distraction histogenesis surgical procedure utilizing the systems and devices described herein. In FIGS. 18A-18E, the method 1800 is being performed on a tibia bone of a patient as an exemplary use-case. It should be appreciated that the systems, methods, and devices described herein may be utilized on alternative bones and tissues as deemed appropriate. The systems described herein may be utilized to perform a periosteal distraction procedure as described in connection with FIGS. 18A-18E. The systems described herein may additionally be utilized to perform distraction histogenesis procedures on other tissue types.

The method 1800 may begin with planning a distraction plate position at 1802 and marking the patient's skin to indicate an incision location. This step is only necessary if the healthcare provider determines that a distraction plate (see 106) would improve patient outcomes based on the patient's unique health circumstances. In some cases, the healthcare provider may determine that a distraction plate (see 106) is not necessary, and that the patient may benefit from only the use of an anchor screw (see 102) and actuation screw (see 104) for distracting and retracting the patient's tissues such as bone or periosteum.

The method 1800 includes creating at 1804 a longitudinal or transverse skin incision and a transverse incision of the patient's periosteum layer. The method 1800 includes inserting at 1806 a periosteal elevator to prepare subperiosteal tunnel on both sides of the incision. The method 1800 is such that on a distal end of the patient's bone, the periosteal elevator is inserted for a whole length of the distraction plate (see 106). The method 1800 is such that on a proximal end of the patient's bone, the periosteal elevator is inserted for a half-length of the distraction plate (see 106). The method 1800 includes inserting at 1812 the entire distraction plate (see 106) under the periosteum on the distal side. The method 1800 includes pulling at 1814 the distraction plate (see 106) back under the periosteum on the proximal side. The method 1800 include aligning at 1816 the central threaded hole of the distraction plate with the incision through the periosteum. The method 1800 includes inserting at 1818 a drill sleeve (see 1700) into the central threaded hole of the distraction plate. The method 1800 includes drilling at 1820 a hole into the patient's bone, wherein a drill bit is disposed through the drill sleeve. The method 1800 includes removing at 1822 the drill sleeve and inserting the anchor screw (see 102) through the central threaded hole of the distraction plate (see 106) and into the pilot hole drilled into the patient's bone. The method 1800 includes simultaneously driving at 1824 the anchor screw (see 102) into both the bone and the distraction plate (see 106) using cannulated driver (see 2400) until the anchor screw (see 102) is locked flush into the distraction plate (see 106). The method 1800 includes inserting at 1826 the actuator linkage (see 108) into the socket head of the anchor screw. The method 1800 includes attaching at 1828 the knob (see 1102) or automated fixator adjustor (see 1502) on to the top plate of the actuator linkage (see 108). The method 1800 includes rotating at 1830 the knob (see 1102) or the automated fixator adjustor (see 1502) to distract or retract the actuation screw (see 104), and accordingly lift up or relax the patient's periosteum.

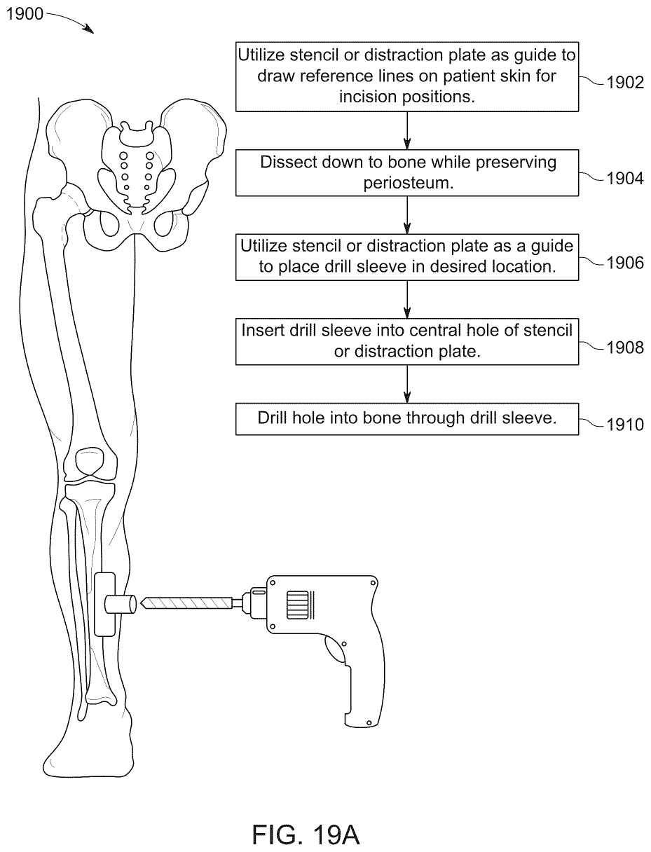

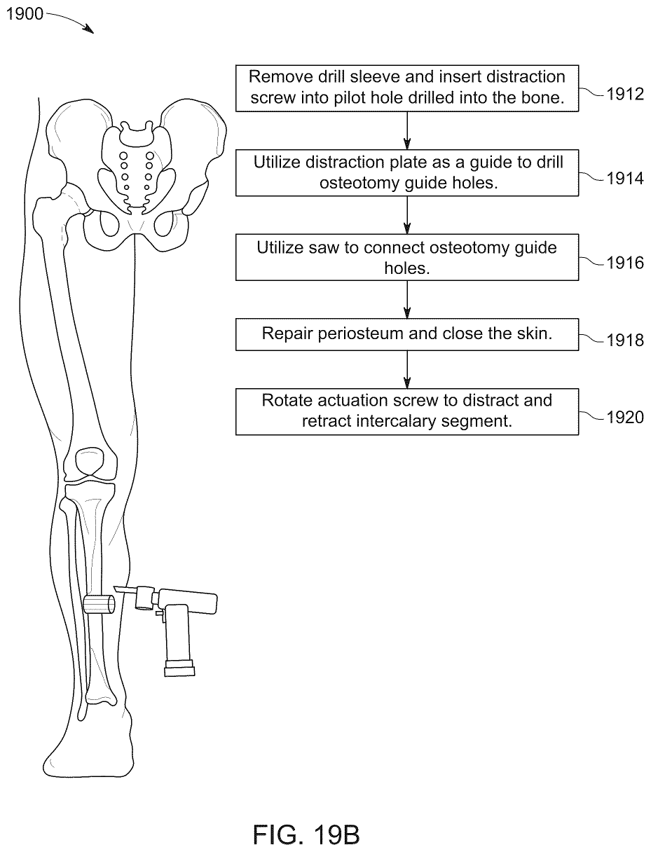

FIGS. 19A-19B are schematic block diagrams of a method 1900 for performing a distraction histogenesis surgical procedure utilizing the systems and devices described herein, and specifically for performing an osteogenesis surgical procedure. In FIGS. 19A-19B, the method 1900 is being performed on a tibia bone of a patient as an exemplary use-case. It should be appreciated that the systems, methods, and devices described herein may be utilized on alternative bones and tissues as deemed appropriate. The systems described herein may additionally be utilized to perform distraction histogenesis procedures on other tissue types.

The method 1900 may begin with utilizing at 1902 a stencil or distraction plate as a guide to draw reference lines on patient skin for incision positions. The method 1900 includes dissecting at 1904 down to bone while preserving the periosteum. The method 1900 includes utilizing at 1906 the stencil or the distraction plate as a guide to place a drill sleeve in the desired location. The method 1900 includes inserting at 1908 the drill sleeve into the central hole of the stencil or distraction plate. The method 1900 includes drilling at 1910 a hole into the bone through the drill sleeve.

The method 1900 includes removing at 1912 the drill sleeve and inserting a distraction screw (including an anchor screw and actuation screw) into pilot hole drilled into the bone. The method 1900 includes utilizing at 1914 the stencil or distraction plate as a guide to drill osteotomy guide holes. The method 1900 includes utilizing at 1916 a saw to connect the osteotomy guide holes. The method 1900 includes repairing the periosteum at 1918 and closing the skin. The method 1900 includes rotating at 1920 the actuation screw to distract and retract the intercalary segment.

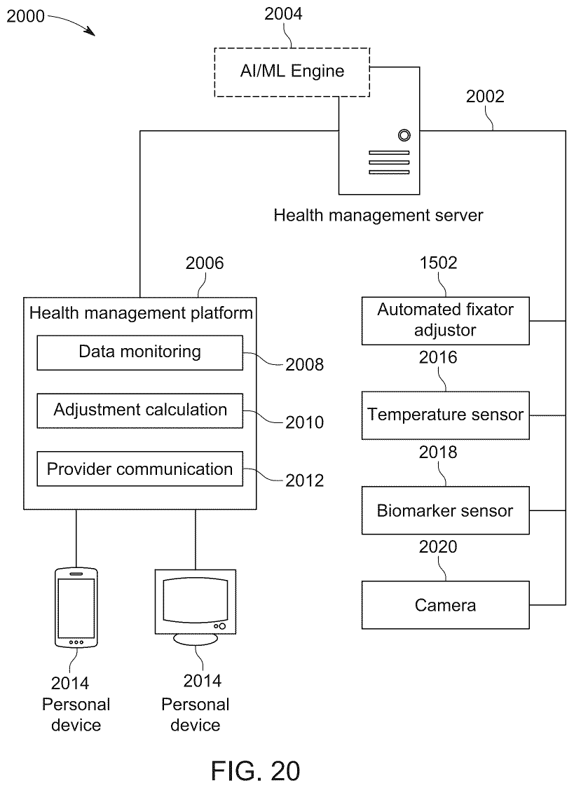

FIG. 20 is a schematic block diagram of a system 2000 for remotely monitoring and controlling a distraction device. The system 2000 may be utilized in connection with any of the systems, methods, or devices described herein. The system 2000 may specifically be utilized in connection with any of the distraction devices described herein, including the system 100 first described in connection with FIG. 1, when the system 100 is actuated with an automated fixator adjustor. The system 2000 may further be used in connection with the system 1500 first described in connection with FIG. 15. The system 2000 may be utilized in connection with a manual fixator adjustor or an automated fixator adjustor 1502.

The system 2000 includes a health management server 2002 that processes operations for a health management platform 2006. The health management server 2002 renders a graphical user interface (GUI) that is made accessible by way of the health management platform 2006. The health management platform 2006 is made accessible to one or more personal devices 2014 by way of a computer-executed application, web browser, or other means. The health management platform 2006 may be accessed with a personal device 2014 that is in communication with a network, such as wide area network, local area network, LoRaWAN, the Internet, and so forth.

The health management server 2002 may optionally include an artificial intelligence and/or machine learning (AI/ML) engine 2004. The AI/ML engine 2004 is trained upon a set of training data. The AI/ML engine 2004 may be trained to assess data output from any of the automated fixator adjustor 1502, a temperature sensor 2016, a biomarker sensor 2018, or a camera 2020 to determine whether there is a likely issue with the patient. The temperature sensor 2016, biomarker sensor 2018, and/or camera 2020 may be implemented as separate components from the automated fixator adjustor 1502 or be integrated and part of the automated fixator adjustor 1502. The AI/ML engine 2004 may further be trained on data from the automated fixator adjustor 1502 or from numerous orthopedic procedures, including osteotomy and distraction histogenesis procedures, to generate a proposed treatment protocol for adjusting a fixator with the automated fixator adjustor 1502.

The health management platform 2006 may include modules for providing information regarding data monitoring 2008, adjustment calculation 2010, provider communication 2012, and so forth. The health management platform 2006 may be rendered on an application that is run on a personal device 2014, such as a mobile phone, a tablet, or other personal computer. The health management platform 2006 may be rendered on a web browser that is accessible by way of a personal device 2014 connected to the Internet or LoRaWAN.

Patients, healthcare providers, administrators, and other users may access the health management platform 2006 with a personal device 2014 to view up-to-date data provided by any of the automated fixator adjustor 1502, temperature sensor 2016, biomarker sensor 2018, or camera 2020. Additionally, the health management platform 2006 provides current and past information regarding adjustment calculations 2010 for a fixator that is currently or was previously fixated to a patient. The adjustment calculations 2010 may be utilized to change or maintain protocols to be implemented by the automated fixator adjustor 100. The health management platform 2012 provides a means for secure bidirectional communication with patients and healthcare providers by way of the provider communication 2012 module. The health management platform 2012 additionally provides a means for secure bidirectional communication with devices such as the temperature sensor 2016, biomarker sensor 2018, camera 2020, and automated fixator adjustor 1502.

The automated fixator adjustor 1502 comprises one or more processors configured to execute instructions stored in non-transitory computer readable storage medium. The processors of the automated fixator adjustor 1502 may receive instructions from any of the health management server 2002, a locally stored memory device with the automated fixator adjustor 1502, or from another source such as the personal device 2014. The automated fixator adjustor 1502 may automatically lift or lower a compliant elongated member of a distraction device to make real-time adjustments in accordance with a healthcare provider's protocol. A healthcare provider may amend the adjustment protocol by communicating with the health management server 2002 by way of the health management platform 2006.

The automated fixator adjustor 1502 may include one or more Bluetooth® low energy chips and/or near-field communication chips that enable the automated fixator adjustor 1502 to wirelessly communicate with personal devices 2014 by way of Bluetooth® or near-field communication. The automated fixator adjustor 1502 may further be equipped with Wi-Fi® or LoRaWAN capability that enables the automated fixator adjustor 1502 to wirelessly communicate with the health management server 2002 directly.

The temperature sensor 2016 may be temporarily installed within a patient to monitor the real-time internal temperature of the patient. Data output by the temperature sensor 2016 may be assessed to determine if patient tissue is inflamed or if the patient may have an infection at the surgical site or there is enough blood flow on extremities of patient's body.

The biomarker sensor 2018 may be temporarily installed within a patient to monitor the real-time presence of certain biomarkers. The biomarker sensor 2018 may specifically be tuned to monitor the presence of biomarkers that may signal the patient currently has an infection at the surgical site. The data output by the biomarker sensor 2018 may be utilized to determine if the patient likely has an infection at the surgical site and requires additional care. The biomarker sensor 2018 may additionally be utilized to detect and measure growth factors or positive biomarkers associated with patient healing.

The camera 2020 may include a standalone camera or one or more image sensors, light sources, photocells, other sensors, and other features associated with another computing device such as a mobile phone. The camera 2020 may additionally or alternatively include a spectroscopy system, including, for example a Near Infrared Spectroscopy (NIRS) system utilized for measuring tissue oxygenation. The camera 2020 may thus be utilized to measure progress of perfusion to tissues. In some cases, a personal device 2014 comprising a camera communicates with the health management server 2002 by way of a computer-executed application. The health management server 2002 executes the application and may communicate directly with the camera 2020 to receive images and other data captured by the camera 2020. In some cases, the camera 2020 may integrated with any of the systems described herein and may capture up-to-date pictures of a fixator installed on a patient.

The personal device 2014 is any personal computing device that can communicate with the health management server 2002. The personal device 2014 may include a smart phone, a tablet, a laptop, a personal computer, virtual or augmented reality device, and so forth. The personal devices 2014 may communicate with the health management server 2002 by way of a local area network (LAN) connection, a wide area network (WAN) connection, or another network connection, 4G/5G data network, or satellite signals.

The personal device 2014 may include an application installed thereon for enabling streamlined 2-way communication with the health management server 2002. The application is configured for multiple platforms, including, for example, smart phone platforms, tablet platforms, Android® operating systems, Linux® operating systems, Windows® operating systems, iOS® operating systems, Macintosh® operating systems, and so forth. The application is configured to provide secure bidirectional communication with one or more of the health management server 2002, the temperature sensor 2016, the biomarker sensor 2018, the camera 2020, or the automated fixator adjustor 1502. The application may wirelessly receive sensor information, system status information, and may further send commanding signals to sensors and system components. The application is configured to display system information through one or more means, including, for example, graphic displays, audible sounds, videos, and so forth. The application is configured to receive user input and is capable of communicating with multiple systems.

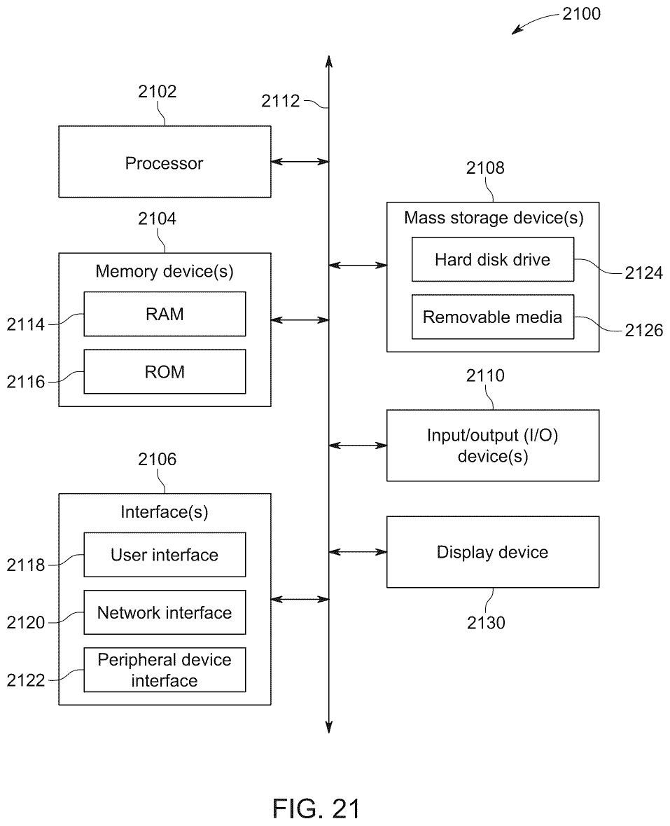

Referring now to FIG. 21, a block diagram of an example computing device 2100 is illustrated. Computing device 2100 may be used to perform various procedures, such as those discussed herein. Computing device 2100 can perform various monitoring functions as discussed herein, and can execute one or more application programs, such as the application programs or functionality described herein. Computing device 2100 can be any of a wide variety of computing devices, such as a desktop computer, in-dash computer, vehicle control system, a notebook computer, a server computer, a handheld computer, tablet computer and the like. The computing device 2100 described herein can also embedded as part of the automated fixator adjustor 1502 instead of a separate standalone device.

Computing device 2100 includes one or more processor(s) 2112, or microcontrollers, one or more memory device(s) 2104, one or more interface(s) 2106, one or more mass storage device(s) 2108, one or more Input/output (I/O) device(s) 2110, and a display device 2130 all of which are coupled to a bus 2112. Processor(s) 2112 include one or more processors or controllers that execute instructions stored in memory device(s) 2104 and/or mass storage device(s) 2108. Processor(s) 2112 may also include diverse types of computer-readable media, such as cache memory.

Memory device(s) 2104 include various computer-readable media, such as volatile memory (e.g., random access memory (RAM) 2114) and/or nonvolatile memory (e.g., read-only memory (ROM) 2116). Memory device(s) 2104 may also include rewritable ROM, such as Flash memory.

Mass storage device(s) 2108 include various computer readable media, such as magnetic tapes, magnetic disks, optical disks, solid-state memory (e.g., Flash memory), and so forth. As shown in FIG. 21, a particular mass storage device 2108 is a hard disk drive 2124. Various drives may also be included in mass storage device(s) 2108 to enable reading from and/or writing to the various computer readable media. Mass storage device(s) 2108 include removable media 2126 and/or non-removable media.

I/O device(s) 2110 include various devices that allow data and/or other information to be input to or retrieved from computing device 2100. Example I/O device(s) 2110 include cursor control devices, keyboards, keypads, microphones, monitors, touchscreen devices, or other display devices, speakers, printers, network interface cards, modems, and the like.

Display device 2130 includes any type of device capable of displaying information to one or more users of computing device 2100. Examples of display device 2130 include a monitor, display terminal, video projection device, and the like.

Interface(s) 2106 include various interfaces that allow computing device 2100 to interact with other systems, devices, or computing environments. Example interface(s) 2106 may include any number of different network interfaces 2120, such as interfaces to local area networks (LANs), wide area networks (WANs), 4G/5G data network, satellite signals, wireless networks, and the Internet. Other interface(s) include user interface 2118 and peripheral device interface 2122. The interface(s) 2106 may also include one or more user interface elements 2118. The interface(s) 2106 may also include one or more peripheral interfaces such as interfaces for printers, pointing devices (mice, track pad, or any suitable user interface now known to those of ordinary skill in the field, or later discovered), keyboards, and the like.

Bus 2112 allows processor(s) 2112, memory device(s) 2104, interface(s) 2106, mass storage device(s) 2108, and I/O device(s) 2110 to communicate with one another, as well as other devices or components coupled to bus 2112. Bus 2112 represents one or more of several types of bus structures, such as a system bus, PCI bus, IEEE bus, USB bus, and so forth.

For purposes of illustration, programs and other executable program components are shown herein as discrete blocks, although it is understood that such programs and components may reside at various times in different storage components of computing device 1800 and are executed by processor(s) 2112. Alternatively, the systems and procedures described herein can be implemented in hardware, or a combination of hardware, software, and/or firmware. For example, one or more application specific integrated circuits (ASICs) can be programmed to conduct one or more of the systems and procedures described herein. As used herein, the terms “module” or “component” are intended to convey the implementation apparatus for accomplishing a process, such as by hardware, or a combination of hardware, software, and/or firmware, for the purposes of performing all or parts of operations disclosed herein. The terms “module” or “component” are intended to convey independent in how the modules, components, or their functionality or hardware may be implemented in different embodiments.

### EXAMPLES

The following examples pertain to further embodiments.

Example 1 is a system for performing a distraction histogenesis surgical procedure. The system includes an anchor screw comprising a hollow interior defined by a sidewall, wherein the anchor screw comprises external threading attached to an external surface of the sidewall, and wherein the anchor screw comprises internal threading attached to an internal surface of the sidewall. The system includes an actuation screw comprising actuation threading attached to an exterior surface of the actuation screw. The system includes an actuator linkage configured to be coupled to the actuation screw. The system is such that the actuation screw is configured to be disposed within the hollow interior of the anchor screw such that the distraction threading interfaces with the internal threading of the anchor screw. The system is such that rotation of the actuator linkage causes rotation of the actuation screw such that the actuation screw is screwed into and out of the hollow interior of the anchor screw.

Example 2 is a system as in Example 1, further comprising a distraction plate configured to be disposed in between a first tissue and a second tissue of a patient, wherein the actuation screw is coupled to the distraction plate along a longitudinal axis of the actuation screw.

Example 3 is a system as in any of Examples 1-2, wherein the distraction plate comprises a hole defined by a sidewall that comprises internal threading, and wherein the hole is disposed through a thickness of the distraction plate.

Example 4 is a system as in any of Examples 1-3, wherein the internal threading of the sidewall of the hole of the distraction plate corresponds with external threading of a coupler such that the coupler may be screwed into the distraction plate.

Example 5 is a system as in any of Examples 1-4, further comprising an adjustor coupled to the actuator linkage, wherein the adjustor comprises a knob, and wherein the knob is configured to be coupled to the actuator linkage to enable a user to manually rotate the actuator linkage.

Example 6 is a system as in any of Examples 1-5, further comprising an adjustor coupled to the actuator linkage, wherein the adjustor comprises an automated fixator adjustor, and wherein the automated fixator adjustor comprises: a motor or gearmotor; and a driveshaft in mechanical communication with the motor; wherein the drive shaft drives the anchor screw via the actuator linkage.

Example 7 is a system as in any of Examples 1-6, wherein the automated fixator adjustor further comprises: a processor; and a wireless communication antenna or chip in RF communication with the processor; wherein the processor actuates the motor in response to receiving an instruction to actuate the motor, wherein the instruction is received via one or more of preloaded storage or the wireless communication antenna or chip.

Example 8 is a system as in any of Examples 1-7, wherein the anchor screw is configured to be screwed into a bone tissue of a patient, and wherein rotation of the actuator linkage in a first direction causes the actuation screw to lift away from an outer surface of the bone tissue.

Example 9 is a system as in any of Examples 1-8, wherein rotation of the actuator linkage in a second direction that is opposite to the first direction causes the actuation screw to lower toward the outer surface of the bone tissue.

Example 10 is a system as in any of Examples 1-9, wherein the anchor screw is configured to be screwed into a bone tissue of a patient, and wherein the actuator linkage is configured to be disposed through a skin layer and a periosteum layer of the patient.

Example 11 is a system as in any of Examples 1-10, further comprising an adjustor located external to a skin layer of the patient when the system is installed in a patient.

Example 12 is a system as in any of Examples 1-11, wherein the actuator linkage comprises: a top plate, wherein the top plate comprises a top polygonal cross-sectional geometry; a bottom plate, wherein the bottom plate comprises a bottom polygonal cross-sectional geometry; and a shaft disposed in between the top plate and the bottom plate.

Example 13 is a system as in any of Examples 1-12, wherein a length of the shaft is optimized based on a thickness of patient tissue from the outer surface of the bone to an exterior of a skin layer of the patient.

Example 14 is a system as in any of Examples 1-13, wherein one or more of the top polygonal cross-sectional geometry or the bottom polygonal cross-sectional geometry is a hexagonal geometry.

Example 15 is a system as in any of Examples 1-14, further comprising a distraction plate coupled to the actuation screw, wherein the distraction plate comprises an irregular octagonal geometry.

Example 16 is a system as in any of Examples 1-15, wherein the irregular octagonal geometry of the distraction plate comprises two long sides that comprise substantially equivalent length and oriented substantially parallel to one another, and wherein the substantially equivalent length of the two long sides is selected to perform periosteal distraction on a leg bone of a patient.

Example 17 is a system as in any of Examples 1-16, wherein the irregular octagonal geometry of the distraction plate comprises two long sides that comprise substantially equivalent length and oriented substantially parallel to one another, and wherein the substantially equivalent length of the two long sides is selected to perform periosteal distraction on an arm bone of a patient.

Example 18 is a system as in any of Examples 1-17, further comprising a distraction plate coupled to the actuation screw, wherein the distraction plate is configured to be disposed in between a circumferential lamellae of a bone and a periosteum layer of the bone such that a tightening rotation of the actuator linkage causes the distraction plate to lift the periosteum layer away from the circumferential lamellae.

Example 19 is a system as in any of Examples 1-18, further comprising a distraction plate coupled to the actuation screw, wherein the distraction plate is configured to be disposed in between a circumferential lamellae of a bone and the periosteum layer of the bone such that a loosening rotation of the actuator linkage causes the distraction plate to lower toward the circumferential lamellae.

Example 20 is a system as in any of Examples 1-19, further comprising a drill sleeve, wherein the drill sleeve comprises: external threading; and a hollow cylinder; wherein the external threading corresponds with internal threading of a hole disposed through the distraction plate; and wherein a diameter of the hollow cylinder is configured to receive a drill bit for drilling a pilot hole into the bone.

Example 21 is a system as in any of Examples 1-20, further comprising a cannulated driver, wherein the cannulated driver comprises a driver head configured to interface with a socket head of a coupler, wherein the coupler axially couples the distraction plate to the actuation screw without also rotationally coupling the actuation screw to the distraction plate.

Example 22 is a system for performing a distraction histogenesis surgical procedure. The system includes an anchor screw comprising a hollow interior defined by a sidewall, wherein the anchor screw comprises internal actuation threading attached to an interior surface of the sidewall and further comprises external anchor threading attached to an exterior surface of the sidewall. The system includes an actuation screw comprising external actuation threading attached to an exterior surface of the actuation screw. The system includes a distraction plate coupled to the actuation screw. The system is such that the external actuation threading corresponds with the internal actuation threading such that the actuation screw is configured to be screwed into the hollow interior of the anchor screw. The system is such that the distraction plate is rotationally independent of the actuation screw.

Example 23 is a system as in Example 22, wherein the distraction plate is axially coupled to the actuation screw such that: rotation of the actuation screw in a first rotational direction causes the actuation screw to screw into the anchor screw and further causes the distraction plate to lower toward the anchor screw; and rotation of the actuation screw in a second rotational direction causes the actuation screw to screw out of the anchor screw and further causes the distraction plate to lift away from the anchor screw.

Example 24 is a system as in any of Examples 22-23, wherein the distraction plate is rotationally independent of the actuation screw such that the distraction plate remains rotationally stationary in response to the rotation of the actuation screw in either of the first rotational direction or the second rotational direction.

Example 25 is a system as in any of Examples 22-24, further comprising a coupler that couples the distraction plate to the actuation screw such that the distraction plate is rotationally independent of the actuation screw, wherein the coupler comprises a sidewall that defines a hollow interior, and wherein the sidewall comprises: an anchoring interface portion that comprises external threading attached to an exterior surface of the sidewall; and a rotational interface portion attached to the anchoring interface portion.

Example 26 is a system as in any of Examples 22-25, wherein the sidewall of the coupler comprises a polygonal cross-sectional geometry at the rotational interface portion such that the rotational interface portion forms a polygonal socket driver head having a hollow interior.

Example 27 is a system as in any of Examples 22-26, wherein the anchor screw further comprises a polygonal socket interface comprising a polygonal cross-sectional geometry; and wherein the polygonal socket driver head of the rotational interface is configured to interface with the polygonal socket interface of the anchor screw to drive rotation of the anchor screw.

Example 28 is a system as in any of Examples 22-27, wherein the distraction plate comprises a threaded hole disposed therethrough; and wherein the external threading attached to the exterior surface of the coupler is configured to interface with corresponding internal threading disposed on the threaded hole of the distraction plate.

Example 29 is a system as in any of Examples 22-28, wherein the actuation screw comprises an outer screw shaft forming the exterior surface of the actuation screw, wherein the outer screw shaft comprises: a sidewall that defines an at least partially hollow interior, wherein the sidewall defines the exterior surface of the actuation screw and further defines an interior surface of the actuation screw; the external actuation threading attached to the exterior surface of the actuation screw; and internal coupling threading attached to the interior surface of the actuation screw.

Example 30 is a system as in any of Examples 22-29, wherein the actuation screw further comprises an inner screw shaft configured to be disposed within the outer screw shaft and the coupler, wherein the inner screw shaft comprises: a screw head comprising a polygonal cross-sectional geometry; a smooth shaft portion attached to the screw head; and a threaded portion comprising external coupling threading.

Example 31 is a system as in any of Examples 22-30, wherein the external coupling threading is configured to interface with the internal coupling threading to couple the inner screw shaft to the outer screw shaft.

Example 32 is a system as in any of Examples 22-31, wherein the smooth shaft portion of the inner screw shaft is configured to be disposed within the rotational interface portion of the coupler; and wherein an internal diameter of the rotational interface portion is greater than an external diameter of the smooth shaft portion such that the inner screw shaft rotates freely within the coupler.

Example 33 is a system as in any of Examples 22-32, wherein the coupler is configured to axially couple the distraction plate to the actuation screw such that: driving the screw head of the inner screw shaft of the actuation screw in a first rotational direction causes the actuation screw to rotate in the first rotational direction to screw into the anchor screw, and further causes the distraction plate to lower toward the anchor screw; and driving the screw head of the inner screw shaft of the actuation screw in a second rotational direction causes the actuation screw to screw out of the anchor screw, and further causes the distraction plate to lift away from the anchor screw; wherein the distraction plate is rotationally independent of the actuation screw such that the distraction plate remains rotationally stationary in response to the rotation of the actuation screw in either of the first rotational direction or the second rotational direction.

Example 34 is a system as in any of Examples 22-33, wherein the distraction plate is configured to be disposed in between a first tissue and a second tissue of a patient.

Example 35 is a system as in any of Examples 22-34, wherein the actuation screw comprises a screw head comprising a polygonal cross-sectional geometry; wherein rotation of the screw head in a first rotational direction causes the actuation screw to screw into the anchor screw; and wherein rotation of the screw head in a second rotational direction causes the actuation screw to screw out of the anchor screw.

Example 36 is a system as in any of Examples 22-35, further comprising an automated adjustor coupled to the screw head of the actuation screw, wherein the automated adjustor comprises: one or more of a motor or gearmotor; and a driveshaft in mechanical communication with the one or more of the motor or the gearmotor; wherein the drive shaft drives rotation of the screw head.

Example 37 is a system as in any of Examples 22-36, wherein the automated adjustor further comprises: a processor; a motor driver; a power source comprising one or more of a battery or external power; and a wireless communication antenna or chip in communication with the processor; wherein the processor controls the one or more of the motor or the gearmotor via the motor driver in response to receiving an instruction to actuate the one or more of the motor or the gearmotor, and wherein the instruction is received via one or more of preloaded storage or the wireless communication antenna or chip.

Example 38 is a system as in any of Examples 22-37, wherein the external anchor threading of the anchor screw is configured to be screwed into a tissue substrate of a patient; wherein rotation of the actuation screw in a first rotational direction causes the actuation screw to screw into the anchor screw, and further causes the distraction plate to lower toward the anchor screw, without adjusting a position of the anchor screw within the tissue substrate; and wherein rotation of the actuation screw in a second rotational direction causes the actuation screw to screw out of the anchor screw, and further causes the distraction plate to lift away from the anchor screw, without adjusting a position of the anchor screw within the tissue substrate.

Example 39 is a system as in any of Examples 22-38, further comprising an adjustor located external to a skin layer of the patient when the system is installed in a patient.

Example 40 is a system as in any of Examples 22-39, wherein the anchor screw is configured to be screwed into a tissue substrate of a patient; and wherein a position of the actuation screw relative to a longitudinal axis of the anchor screw is adjusted and maintained by rotating the actuation screw within the hollow interior of the anchor screw.

Example 41 is a system as in any of Examples 22-40, further comprising an actuator linkage, wherein the actuator linkage comprises an actuation screw coupling head configured to interface with a screw head of the actuation screw, and wherein rotation of the actuator linkage causes synchronous rotation of the actuation screw when the actuator linkage is coupled to the actuation screw.

Example 42 is a system for performing a distraction histogenesis surgical procedure. The system includes a distraction device comprising: an anchor screw comprising a hollow interior defined by a sidewall, wherein the anchor screw comprises internal actuation threading attached to an interior surface of the sidewall and further comprises external anchor threading attached to an exterior surface of the sidewall; an actuation screw comprising a screw head and external actuation threading attached to an exterior surface of the actuation screw; and a distraction plate coupled to the actuation screw; wherein the external actuation threading corresponds with the internal actuation threading such that the actuation screw is configured to be screwed into the hollow interior of the anchor screw; and wherein the distraction plate is rotationally independent of the actuation screw. The system includes an automated adjustor coupled to the screw head of the actuation screw and configured to drive rotation of the actuation screw, wherein the automated adjustor comprises: one or more of a motor or a gearmotor; a motor driver; a power source comprising one or more of a battery or an external power source; a driveshaft in mechanical communication with the one or more of the motor or the gearmotor; and a processor, wherein the processor controls the one or more of the motor or the gearmotor based upon a computer-readable instruction via the motor driver.

The foregoing description has been presented for purposes of illustration. It is not exhaustive and does not limit the invention to the precise forms or embodiments disclosed. Modifications and adaptations will be apparent to those skilled in the art from consideration of the specification and practice of the disclosed embodiments. For example, components described herein may be removed and other components added without departing from the scope or spirit of the embodiments disclosed herein or the appended claims.

Other embodiments will be apparent to those skilled in the art from consideration of the specification and practice of the disclosure disclosed herein. It is intended that the specification and examples be considered as exemplary only, with a true scope and spirit of the invention being indicated by the following claims.

What is claimed is:

1. A system for performing a distraction histogenesis surgical procedure, the system comprising:
an anchor screw comprising a hollow interior defined by a sidewall, wherein the anchor screw comprises internal actuation threading attached to an interior surface of the sidewall and further comprises external anchor threading attached to an exterior surface of the sidewall;
an actuation screw comprising external actuation threading attached to an exterior surface of the actuation screw; and
a distraction plate coupled to the actuation screw;
wherein the external actuation threading corresponds with the internal actuation threading such that the actuation screw is configured to be screwed into the hollow interior of the anchor screw; and
wherein the distraction plate is rotationally independent of the actuation screw.

2. The system of claim 1, wherein the distraction plate is axially coupled to the actuation screw such that:
rotation of the actuation screw in a first rotational direction causes the actuation screw to screw into the anchor screw and further causes the distraction plate to lower toward the anchor screw; and
rotation of the actuation screw in a second rotational direction causes the actuation screw to screw out of the anchor screw and further causes the distraction plate to lift away from the anchor screw.

3. The system of claim 2, wherein the distraction plate is rotationally independent of the actuation screw such that the distraction plate remains rotationally stationary in response to the rotation of the actuation screw in either of the first rotational direction or the second rotational direction.

4. The system of claim 1, further comprising a coupler that couples the distraction plate to the actuation screw such that the distraction plate is rotationally independent of the actuation screw, wherein the coupler comprises a sidewall that defines a hollow interior, and wherein the sidewall comprises:
an anchoring interface portion that comprises external threading attached to an exterior surface of the sidewall; and
a rotational interface portion attached to the anchoring interface portion.

5. The system of claim 4, wherein the sidewall of the coupler comprises a polygonal cross-sectional geometry at the rotational interface portion such that the rotational interface portion forms a polygonal socket driver head having a hollow interior.

6. The system of claim 5, wherein the anchor screw further comprises a polygonal socket interface comprising a polygonal cross-sectional geometry; and
wherein the polygonal socket driver head of the rotational interface is configured to interface with the polygonal socket interface of the anchor screw to drive rotation of the anchor screw.

7. The system of claim 4, wherein the distraction plate comprises a threaded hole disposed therethrough; and
wherein the external threading attached to the exterior surface of the coupler is configured to interface with corresponding internal threading disposed on the threaded hole of the distraction plate.

8. The system of claim 4, wherein the actuation screw comprises an outer screw shaft forming the exterior surface of the actuation screw, wherein the outer screw shaft comprises:
a sidewall that defines an at least partially hollow interior, wherein the sidewall defines the exterior surface of the actuation screw and further defines an interior surface of the actuation screw;
the external actuation threading attached to the exterior surface of the actuation screw; and
internal coupling threading attached to the interior surface of the actuation screw.

9. The system of claim 8, wherein the actuation screw further comprises an inner screw shaft configured to be disposed within the outer screw shaft and the coupler, wherein the inner screw shaft comprises:
a screw head comprising a polygonal cross-sectional geometry;
a smooth shaft portion attached to the screw head; and
a threaded portion comprising external coupling threading.

10. The system of claim 9, wherein the external coupling threading is configured to interface with the internal coupling threading to couple the inner screw shaft to the outer screw shaft.

11. The system of claim 9, wherein the smooth shaft portion of the inner screw shaft is configured to be disposed within the rotational interface portion of the coupler;
wherein an internal diameter of the rotational interface portion is greater than an external diameter of the smooth shaft portion such that the inner screw shaft rotates freely within the coupler.

12. The system of claim 11, wherein the coupler is configured to axially couple the distraction plate to the actuation screw such that:
driving the screw head of the inner screw shaft of the actuation screw in a first rotational direction causes the actuation screw to rotate in the first rotational direction to screw into the anchor screw, and further causes the distraction plate to lower toward the anchor screw; and
driving the screw head of the inner screw shaft of the actuation screw in a second rotational direction causes the actuation screw to screw out of the anchor screw, and further causes the distraction plate to lift away from the anchor screw;
wherein the distraction plate is rotationally independent of the actuation screw such that the distraction plate remains rotationally stationary in response to the rotation of the actuation screw in either of the first rotational direction or the second rotational direction.

13. The system of claim 1, wherein the distraction plate is configured to be disposed in between a first tissue and a second tissue of a patient.

14. The system of claim 1, wherein the actuation screw comprises a screw head comprising a polygonal cross-sectional geometry;
wherein rotation of the screw head in a first rotational direction causes the actuation screw to screw into the anchor screw; and
wherein rotation of the screw head in a second rotational direction causes the actuation screw to screw out of the anchor screw.

15. The system of claim 14, further comprising an automated adjustor coupled to the screw head of the actuation screw, wherein the automated adjustor comprises:
one or more of a motor or gearmotor; and
a driveshaft in mechanical communication with the one or more of the motor or the gearmotor;
wherein the drive shaft drives rotation of the screw head.

16. The system of claim 15, wherein the automated adjustor further comprises:
a processor;
a motor driver;
a battery or an external power source; and
a wireless communication antenna or chip in communication with the processor;
wherein the processor actuates the one or more of the motor or the gearmotor via the motor driver in response to receiving an instruction to actuate the one or more of the motor or the gearmotor, and wherein the instruction is received via one or more of preloaded storage or the wireless communication antenna or chip.

17. The system of claim 1, wherein the external anchor threading of the anchor screw is configured to be screwed into a tissue substrate of a patient;
wherein rotation of the actuation screw in a first rotational direction causes the actuation screw to screw into the anchor screw, and further causes the distraction plate to lower toward the anchor screw, without adjusting a position of the anchor screw within the tissue substrate; and
wherein rotation of the actuation screw in a second rotational direction causes the actuation screw to screw out of the anchor screw, and further causes the distraction plate to lift away from the anchor screw, without adjusting a position of the anchor screw within the tissue substrate.

18. The system of claim 1, further comprising an adjustor located external to a skin layer of the patient when the system is installed in a patient.

19. The system of claim 1, wherein the anchor screw is configured to be screwed into a tissue substrate of a patient; and
wherein a position of the actuation screw relative to a longitudinal axis of the anchor screw is adjusted and maintained by rotating the actuation screw within the hollow interior of the anchor screw.

20. The system of claim 1, further comprising an actuator linkage, wherein the actuator linkage comprises an actuation screw coupling head configured to interface with a screw head of the actuation screw, and wherein rotation of the actuator linkage causes synchronous rotation of the actuation screw when the actuator linkage is coupled to the actuation screw.

21. A system for performing a distraction histogenesis surgical procedure, the system comprising:
a distraction device comprising:
an anchor screw comprising a hollow interior defined by a sidewall, wherein the anchor screw comprises internal actuation threading attached to an interior surface of the sidewall and further comprises external anchor threading attached to an exterior surface of the sidewall;
an actuation screw comprising a screw head and external actuation threading attached to an exterior surface of the actuation screw; and
a distraction plate coupled to the actuation screw;
wherein the external actuation threading corresponds with the internal actuation threading such that the actuation screw is configured to be screwed into the hollow interior of the anchor screw; and
wherein the distraction plate is rotationally independent of the actuation screw; and
an automated adjustor coupled to the screw head of the actuation screw and configured to drive rotation of the actuation screw, wherein the automated adjustor comprises:
one or more of a motor or a gearmotor;
a driveshaft in mechanical communication with the one or more of the motor or the gearmotor; and
a processor;
a motor driver;
a battery or an external power source; and
wherein the processor actuates the one or more of the motor or the gearmotor based upon a computer-readable instruction.
